(19) United States
(12) Patent Application Publication
(10) Pub. No.: US 2005/0191342 A1
(43) Pub. Date: Sep. 1, 2005

(54) Methods and compositions for enhancing innate immunity and antibody dependent cellular cytotoxicity

(75) Inventors: Ying K. Tam, Vancouver (CA); Ghania Chikh, Vancouver (CA); Laura Sekirov, Richmond (CA); Irina Brodsky, Vancouver (CA); Sameersingh G. Raney, Vancouver (CA)

Correspondence Address: Todd A. Lorenz;Dorsey & Whitney LLP, Intellectual Property Department, Four Embarcadero Center, Suite 3400, San Francisco, CA, 94111-4187, US

(73) Assignee: Inex Pharmaceuticals Corporation

(21) Appl. No.: 10/963,999

(22) Filed: Oct. 12, 2004

Related U.S. Application Data
(60) Provisional application No. 60/616,161, filed on Oct. 4, 2004.
(60) Provisional application No. 60/542,754, filed on Feb. 6, 2004.
(60) Provisional application No. 60/510,799, filed on Oct. 11, 2003.

(51) Int. Cl. A61K 48/00; A61K 39/395; A61K 9/127

(52) U.S. Cl. 424/450; 514/44; 424/155.1

(57) ABSTRACT

Cationic liposomes with immunostimulatory nucleic acids are shown to stimulate the innate immune response, and synergistic combinations of such liposomal nucleic acids and therapeutic antibodies are provided to dramatically improve antibody dependent cellular cytotoxicity and target cell lysis.

### CROSS-REFERENCE TO RELATED APPLICATIONS

This application claims priority to U.S. Provisional Patent Application Ser. No. [to be assigned], filed Oct. 4, 2004; and to U.S. Provisional Patent Application Ser. No. 60/542,754, filed Feb. 6, 2004; and to U.S. Provisional Patent Application Ser. No. 60/510,799 filed Oct. 11, 2003.

### TECHNICAL FIELD

The present invention relates to antibody therapeutics, and more specifically, to enhancing their efficacy by stimulating the innate immune response.

### BACKGROUND OF THE INVENTION

Innate immunity refers to those immune responses that occur rapidly after infection or development of cancer. They are initiated without prior sensitization to the pathogen or malignant cell, are not antigen specific and are mediated directly by phagocytic cells such as macrophages, cytotoxic cells such as natural killer (NK) cells and antigen presenting cells such as dendritic cells (DCs) as well as indirectly by the cytokines produced by these cells. Adaptive immunity refers to those responses that require some time to develop after initial infection or cancer development and involves an education of immune cells, resulting in the development of a highly specific, highly potent and long-lived response. This is mediated by cytotoxic T-lymphocytes (CTLs), helper T-lymphocytes and antibody-producing B-lymphocytes. Along these lines, adaptive immune responses are classified as either cellular (those mediated by CTLs) or humoral (antibody mediated responses), with helper T-lymphocytes facilitating both responses. Together, the rapid innate immune response functions to control early spread of the disease and facilitates development of adaptive immune responses while the highly potent, specific and long-lived adaptive response serves to clear the disease as well as to protect against recurrence.

Although innate and adaptive immunity are often thought of as independent phenomena, there are many bridges connecting them including, e.g., the fact that innate immune responses initiate adaptive immunity. Another way in which they are related is through a process known as antibody-dependent cellular cytotoxicty (“ADCC”). ADCC involves the process by which cells of the innate immune system, predominantly NK cells and macrophages, are able to specifically recognize and attack target cells that have antibodies bound to their surface (i.e. opsonized cells). This is mediated through the presence of specific receptors on these innate immune cells that recognize and bind the Fc region of antibodies. This binding allows recognition of target cells and also triggers the cytolytic mechanisms of the cells leading to target cell killing.

A promising and rapidly developing area of cancer immunotherapy focuses on harnessing immune effector mechanisms for purposes of tumor eradication, utilizing monoclonal antibodies directed to tumor-associated antigens. Advances in the humanization of murine-derived antibodies have greatly improved the utility of these molecules as therapeutics, by reducing or substantially eliminating adverse immune reactions directed against the molecules. Exemplary among these are Herceptin™, the CDR-grafted anti-Her2/neu antibody developed for metastatic breast cancer, and Rituxan™, a chimeric anti-CD20 antibody for Non-Hodgkin's lymphoma.

While the specific modes of action differ for each antibody, they are generally a combination of direct (e.g. blocking engagement of a cell surface receptor necessary for growth or survival or direct induction of apoptosis) and indirect (e.g. induction of immune mediated responses) effects. These immune-mediated effects specifically include mediation of ADCC and induction of complement-mediated lysis. Unfortunately, while mAbs have the advantage of very high specificity, they exhibit limited potency. Thus, there remains a significant need in the art to improve the efficacy of this class of therapeutics by enhancing the potency of the anti-tumor immune responses generated by their administration.

### SUMMARY OF THE RELEVANT LITERATURE

Oligonucleotides containing one or more unmethylated CpG dinucleotide motifs have been described in the art as effective immune stimulators. See, e.g., U.S. Pat. Nos. 6,194,388; 6,207,646; 6,239,116; 6,653,292; 6,429,199; and 6,426,334. In general, the potential immune stimulatory properties of methylated CpG oligonucleotides have been largely overlooked due to the perception that the higher frequency of methylated cytosine residues found in vertebrate DNA would prevent methylated CpGs from eliciting a meaningful response in vertebrate immune systems. See Messina et al., J. Immunol. 147:1759 (1991). Although some reports have emerged of free methylated CpG oligonucleotides having potential immune stimulatory properties, an analysis of their underlying data does not support their broad assertions of activity. See International Pub. No. WO 02/069369 (data demonstrates limited activity of oligonucleotide having multiple methylated CpGs). More recently, lipid encapsulation of such oligonucleotides has been shown to improve their immune stimulatory properties in adaptive immune responses. See, e.g., co-pending U.S. patent application Ser. No. 10/437,275.

### SUMMARY OF THE INVENTION

It has now been surprisingly discovered that the efficacy of antibody therapeutics in effectuating lysis of target cells can be dramatically improved by administering the antibodies in conjunction with cationic liposomes comprising immunostimulatory nucleic acids. As demonstrated for the first time herein, the coadministration of therapeutic antibodies and such liposomal nucleic acids provides a strong synergistic improvement in target cell lysis. Without being bound by theory, it appears that the delivery of the immunostimulatory nucleic acids by the cationic liposome results in a significant enhancement of the innate immune response, and antibody dependent cellular cytotoxicity in particular.

In one aspect, therefore, the invention provides compositions for enhancing antibody dependent cellular cytotoxicity and mediating lysis of target cells in a subject, comprising a therapeutic antibody and a cationic liposome comprising an immunostimulatory nucleic acid, preferably an oligodeoxynucleotide (ODN), more preferably an ODN comprising at least one CpG motif, and most preferably an ODN comprising at least one methylated CpG. In a specific embodiment, the ODN comprises the nucleic acid sequence 5′ TAAZGTTGAGGGGCAT 3′ (ODN1m) (SEQ ID NO:4). In another specific embodiment, the ODN comprises the nucleic acid sequence 5′ TTCCATGAZGTTCCTGAZGTT 3′ (ODN2m) (SEQ ID NO:31). The nucleic acid may be either complexed with or encapsulated by the cationic liposome, and preferably is fully encapsulated by the cationic liposome.

The therapeutic antibody may be monoclonal or polyclonal and may be directed at any target antigen of interest, including pathogen antigens and tumor-associated antigens. Preferred therapeutic antibodies for use in the compositions and methods described herein include anti-CD20 antibodies (e.g., Rituxan™, Bexxar™, Zevalin™), anti-Her2/neu antibodies (e.g., Herceptin™), anti-CD33 antibodies (e.g., Mylotarg™), anti-CD52 antibodies (e.g., Campath™), anti-CD22 antibodies, anti-EGF-R antibodies (Erbitux™), anti-HLA-DR10β antibodies, anti-MUC1 antibodies, anti-T cell antibodies (Thymoglobulin™, Simulect™, OKT3™), and the like.

In another aspect, the invention provides improved methods of inducing antibody dependent cellular cytotoxicity against a target cell in a mammalian subject, comprising activating the subject's NK cells ex vivo or in vivo with a cationic liposome comprising an immunostimulatory nucleic acid, and preferably a methylated oligodeoxynucleotide, and opsonizing the target cell in vivo with a therapeutic antibody directed against a target cell antigen; wherein the activated NK cells bind to the Fc portion of the therapeutic antibody in vivo. In a preferred embodiment, the target cell is a tumor cell and the target cell antigen is a tumor-associated antigen.

Also provided are methods for lysing tumor cells, comprising administering to a patient having said tumor cells a therapeutic antibody and a cationic liposome comprising an immunostimulatory nucleic acid, and preferably an oligodeoxynucleotide having at least one methylated CpG dinucleotide, wherein the therapeutic antibody binds to a surface membrane antigen associated with the tumor cell and the cationic liposome mobilizes and activates the patient's NK cells in vivo for effectuating antibody dependent cellular cytotoxicity. The therapeutic antibody and cationic liposome may be administered simultaneously or sequentially. In a particularly preferred embodiment, the cationic liposome is administered prior to the antibody.

Also provided herein are improved methods for treating a cancer patient with monoclonal antibodies directed to tumor-associated antigens, the improvement comprising the pretreatment of the patient with a cationic liposome comprising an immunostimulatory nucleic acid, and preferably a methylated oligodeoxynucleotide, wherein the pretreatment results in the mobilization and activation of patient NK cells for effectuating antibody dependent cellular cytotoxicity. In a specific embodiment, the cancer is lymphoma and the monoclonal antibody is rituximab. In another specific embodiment, the cancer is breast cancer and the therapeutic antibody is trastuzumab.

In a further aspect, the invention provides kits for mediating lysis of target cells in a subject, comprising a therapeutic antibody directed to a target cell antigen and a cationic liposome comprising an immunostimulatory nucleic acid, preferably an oligodeoxynucleotide (ODN), more preferably an ODN comprising at least one CpG motif, and most preferably an ODN comprising at least one methylated CpG. The therapeutic antibody and cationic liposomes may be provided in the same or in separate vials. In a preferred embodiment, the target cell is a tumor cell and the target cell antigen is a tumor-associated antigen.

The invention also relates to a therapeutic mammalian NK cell activated ex vivo or in vivo by a cationic liposome comprising an immunostimulatory nucleic acid, and preferably an oligodeoxynucleotide having at least one methylated CpG dinucleotide, wherein said activated NK cell is bound to the Fc region of a therapeutic antibody directed to a tumor-associated antigen. Also provided herein is a means for lysing tumor cells, comprising a therapeutic antibody directed to a tumor associated antigen and an activated mammalian NK cell as described above.

### BRIEF DESCRIPTION OF THE DRAWINGS

FIGS. 1A-B show the effect of immunostimulatory nucleic acids and cationic liposomes comprising an immunostimulatory nucleic acid on NK cell expansion/mobilization in blood and spleen of C3H mice. FIG. 1A shows results for total NK population in spleen cells. FIG. 1B shows the results for blood NK population.

FIGS. 2A-B show the effect of cationic liposomes comprising an immunostimulatory nucleic acid on NK cell activation using a CD69 marker in blood and spleen. FIG. 1A shows results for total NK population in spleen cells. FIG. 1B shows the results for blood NK population.

FIGS. 3A-E show enhanced NK cytolytic activity induced by cationic liposomes comprising an immunostimulatory nucleic acid. FIG. 3A shows the release of Cr from Yac-1 cells in the spleen. FIG. 3B shows the release of Cr from Yac-1 cells in the blood. FIG. 3C shows the release of Cr from Yac-1 cells and P815 cells in the spleen. FIG. 3D shows the release of Cr from Yac-1 cells and P815 cells in the blood. FIG. 3E shows the release of Cr from Yac-1 cells in the presence of isolated NK cells.

FIGS. 4A-B show that NK cells activated by cationic liposomes comprising an immunostimulatory nucleic acid mediate ADCC activity. FIG. 4A shows the release of Cr from Daudi cells in the blood. FIG. 4B shows the release of Cr from Daudi cells in the presence of isolated NK cells.

FIGS. 5A-C illustrate the ability of cationic liposomes comprising an immunostimulatory nucleic acid to enhance NK and ADCC activation in tumor bearing and tumor free mice using both YAC and M14 target cells. FIG. 5A shows activation of splenic and blood NK cells, as measured by in vitro cytotoxicity levels against Yac-1. FIG. 5B shows activation of splenic NK cells, as measured by in vitro cytotoxicity levels against M14 cells. FIG. 5C shows activation of blood NK cells, as measured by in vitro cytotoxicity levels against M14 cells.

FIGS. 6A-B show the dose response data for cationic liposomes comprising an immunostimulatory nucleic acid relating to enhanced NK activity vs YAC-1 cells in spleen and blood. FIG. 6A shows the activity of spleen NK cells. FIG. 6B shows the activity of blood NK cells.

FIGS. 7A-B show the dose response data for cationic liposomes comprising an immunostimulatory nucleic acid relating to enhanced ADCC activity against Daudi cells in the presence of an anti-CD20 Ab in spleen and blood. FIG. 7A shows ADCC activity in the spleen cells. FIG. 7B shows the ADCC activity in the blood.

FIGS. 8A-B show the effect of a single vs multiple dosing regimen on ADCC activity against Daudi cells in the presence of Rituxan™ in the spleen and blood.

FIGS. 9A-B show the effect of a single vs double dosing regimen on NK and ADCC activity against Daudi cells in the presence of Rituxan™ in the spleen and blood. FIG. 9A shows ADCC activity in the spleen cells. FIG. 9B shows the ADCC activity in the blood.

FIGS. 10A-B demonstrate the enhanced efficacy of cationic liposomes comprising an immunostimulatory nucleic acid in combination with a therapeutic antibody in a SCID/Namalwa model. FIG. 10A shows survival curves for the treated mice. FIG. 10B shows increase in median life span for the treated mice.

FIGS. 11A-C demonstrate the enhanced efficacy of cationic liposomes comprising an immunostimulatory nucleic acid in combination with a therapeutic antibody in C57BI/6 EL-4 SC and IV tumor models. FIG. 11A shows inhibited tumor growth in treated mice. FIG. 11B shows enhanced survival in treated mice. FIG. 11C shows an increase in median life span for the treated mice.

FIGS. 12A-C demonstrate the ability of cationic liposomes comprising an immunostimulatory nucleic acid to mediate ADCC and facilitate proliferation and mobilization of NK cells using a BrdU incorporation assay. FIG. 12A shows an increase in total NK cells in the blood. FIG. 12B shows an increase in NK cell proliferation. FIG. 12C shows the percentage NK cells due to proliferation as compared to the total number of NK cells present in the blood.

FIGS. 13A-B demonstrate the ability of cationic liposomes comprising an immunostimulatory nucleic acid in combination with Herceptin™ to enhance ADCC. FIG. 13A shows the enhanced anti-tumor efficacy of cationic liposomes comprising an immunostimulatory nucleic acid in combination with Herceptin™. FIG. 13B shows an increase in life span for mice treated with an immunostimulatory nucleic acid in combination with Herceptin™.

FIGS. 14A-B demonstrate the ability of cationic liposomes comprising an immunostimulatory nucleic acid in combination with anti-GD2 to enhance ADCC. FIG. 14A shows the enhanced anti-tumor efficacy of cationic liposomes comprising an immunostimulatory nucleic acid in combination with anti-GD2. FIG. 14B shows tumor regression of the treated mice.

FIGS. 15A-B demonstrate the ability of cationic liposomes comprising an immunostimulatory nucleic acid in combination with anti-GD2 to enhance ADCC and to facilitate development of secondary immune responses. FIG. 15A shows the ability of splenocytes isolated from treated mice to lyse EL-4 tumor cells. FIG. 15B shows the presence of immunoglobulins that bind EL-4 tumor cells in the serum of treated mice.

FIG. 16 shows the inhibition of tumor growth in mice treated with cationic liposomes comprising an immunostimulatory nucleic acid in combination with anti-PS.

FIG. 17 demonstrates an inhibition in tumor growth for mice treated with cationic liposomes comprising an immunostimulatory nucleic acid in combination with anti-PS.

FIG. 18 demonstrates an increase in life span for mice with cancerous tumors treated with cationic liposomes comprising an immunostimulatory nucleic acid in combination with Rituxan™.

FIG. 19 shows that administration of cationic liposomes comprising an immunostimulatory nucleic acid is effective in enhancing the anti-tumor efficacy of Herceptin™ in a xenogeneic tumor model using the human breast cancer cell line MCF-7 in SCID mice.

FIGS. 20A-B show that administration of cationic liposomes comprising an immunostimulatory nucleic acid results in homing of NK cells to sites of tumor. FIG. 20A shows enhanced levels of activated NK cells in C57BI/6 animals bearing a SC solid EL-4 tumor after treatment with cationic liposomes comprising an immunostimulatory nucleic acid. FIG. 20B shows enhanced activation of NK cells within the tumor after administration of cationic liposomes comprising an immunostimulatory nucleic acid.

FIG. 21 shows activation of NK cells and enhanced homing to sites of tumor burden following administration of cationic liposomes comprising an immunostimulatory nucleic acid.

### DETAILED DESCRIPTION OF THE INVENTION

The invention described herein relates to a dramatic improvement in the efficacy of antibody therapeutics, comprising the administration of cationic liposomes comprising immunostimulatory nucleic acids in combination with therapeutic antibodies to significantly increase the antibody dependent cellular cytoxicity (ADCC) response against a desired target cell. As demonstrated herein, administration of the subject cationic liposomes results in the mobilization, expansion and/or activation of natural killer (NK) cells and macrophages, two of the major effector populations responsible for ADCC activity.

Accordingly, in one aspect, the present invention provides methods and compositions for stimulating an immune response to a target antigen, more preferably an innate immune response, and still more preferably an ADCC response. In one embodiment, compositions and methods for inducing the mobilization, expansion and activation of NK cells are provided. As described herein, a dramatic and rapid redistribution of NK cells, particularly from the spleen, is observed after administration of the subject compositions resulting in expansion of the peripheral blood NK compartment where they are available to home and localize to sites of high tumor burden. Coincident with this rapid mobilization and expansion of NK cells was a rapid activation of the NK cell population as determined by both expression of cell activation markers and elevated cytolytic activity. Treatment with the subject compositions resulted in elevated expression of activation markers such as CD69 on the surface of NK cells from peripheral blood and spleen compartments.

As demonstrated herein, the activated NK cells obtained by the subject methods have enhanced cytolytic activity, as determined by activity against a standard target cell YAC-1 in a 4 hr Chromium release cytotoxicity assay, compared to cells from untreated animals. Interestingly, cells activated by the subject lipid formulations did not show enhanced cytotoxicity against the P815 tumor cell line, a target cell often used to detect the enhanced activity in cytokine activated killer cells. This suggests that while both nucleic acids and cytokines activate NK cells and induce higher cytolytic activity, the resultant activities are qualitatively distinct, raising the possibility that these two activation strategies may be complimentary and/or synergistic. Furthermore, these nucleic acid activated immune cells mediated enhanced levels of ADCC activity. While the presence of mAbs directed against antigens found on the surface of tumor cells was found to enhance the ability of immune cells from untreated animals to recognize and kill tumor cells, in vivo administration of the cationic liposome-formulated nucleic acids resulted in a dramatic and synergistic enhancement of ADCC activity by immune cells from various tissue compartments, resulting in elevated cytotoxicity against tumor cells in the presence of mAbs. This enhanced ADCC activity resided almost entirely within the NK cell population as demonstrated by cell separation experiments.

Accordingly, in another aspect, the present invention provides compositions and methods for increasing the activity of antibody therapeutics and thereby increasing their therapeutic efficacy, by enhancing the immune responses mediated by these antibodies, including specifically ADCC. The compositions and methods described herein may increase the activity of antibodies that already act through the ADCC mechanism, add an additional effector mechanism to those antibidies that act via alternative pathways and potentially endow anti-tumor activity on non-therapeutic antibodies. In preferred embodiments, the cationic liposomes comprising immunostimulatory nucleic acids are combined with antibody therapeutics to induce a more potent immune response against the target of the antibody therapeutic. Certain of the compositions employ additional components such as cytokines, additional therapeutic agents and/or other components, but these additional components are not necessary for all applications.

As demonstrated herein, administration of the subject cationic liposomes comprising an immunostimulatory nucleic acid were found to enhance the efficacy of a variety of mAbs in several accepted animal models. In a human xenograft model of SCID mice challenged with the human B-cell lymphoma tumor cell line Namalwa and treated with cationic liposomes comprising an immunostimulatory nucleic acid and various doses of anti-CD20 mAb, treatment with the combination was found to enhance survival of these animals (>270% increase in lifespan or ILS) compared to untreated animals, those treated with mAbs alone (18-31% ILS) or nucleic acid alone (112% increase in lifespan). Similarly, in C57BI/6-EL4 thymoma intravenous and subcutaneous models the combination of lipid formulated nucleic acids and mAb (in this case, specific against the ganglioside GD2) have shown therapeutic advantage over either treatment alone in enhancing survival and inhibiting tumor growth respectively.

The invention provides formulations and methods of use thereof, based on the discovery that nucleic acids, including in particular methylated oligonucleotides, and more particularly nucleic acids bearing a methylated CpG dinucleotide motif can dramatically enhance innate immune responses in vivo by including them in cationic liposomes.

In one embodiment, the immunostimulatory nucleic acid comprises at least one CpG dinucleotide having a methylated cytosine. In a preferred embodiment, the nucleic acid comprises a single CpG dinucleotide, wherein the cytosine in said CpG dinucleotide is methylated. In a specific embodiment, the nucleic acid comprises the sequence 5′ TAACGTTGAGGGGCAT 3′ (ODN1m). In an alternative embodiment, the nucleic acid comprises at least two CpG dinucleotides, wherein at least one cytosine in the CpG dinucleotides is methylated. In a further embodiment, each cytosine in the CpG dinucleotides present in the sequence is methylated. In another specific embodiment, the nucleic acid comprises the sequence 5′ TTCCATGACGTTCCTGACGTT 3′ (ODN2m). In another embodiment, the nucleic acid comprises a plurality of CpG dinucleotides, wherein at least one of said CpG dinucleotides comprises a methylated cytosine. As demonstrated herein, effective stimulation of the innate immune response may be obtained utilizing nucleic acids having only a single CpG dinucleotide with a methylated cytosine, or a plurality of CpG dinucleotides wherein only one or a couple of the cytosines of said CpG dinucleotides are methylated.

Detailed methods of making, using and testing the various formulations of the invention are described hereafter and in the references cited herein, all of which are incorporated by reference.

### ABBREVIATIONS AND DEFINITIONS

The following abbreviations are used herein:
RBC, red blood cells;
DDAB, N,N-distearyl-N,N-dimethylammonium bromide;
DODAC, N,N-dioleyl-N,N-dimethylammonium chloride;
DOPE, 1,2-sn-dioleoylphoshatidylethanolamine;
DOSPA, 2,3-dioleyloxy-N-(2(sperminecarboxamido)ethyl)-N,N-dimethyl-1-propanaminium trifluoroacetate;
DOTAP, 1,2-dioleoyloxy-3-(N,N,N-trimethylamino)propane chloride;
DOTMA, 1,2-dioleyloxy-3-(N,N,N-trimethylamino)propanechloride;
OSDAC, N-oleyl-N-stearyl-N,N-dimethylammonium chloride;
RT, room temperature;
HEPES, 4-(2-hydroxyethyl)-1-piperazineethanesulfonic acid;
FBS, fetal bovine serum;
DMEM, Dulbecco's modified Eagle's medium;
PEG-DMG 3-O-[2′-(w-monomethoxypolyethylene glycol2000) succinoyl]-1,2-dimyrsitoyl-sn-glycerol
PEG-Cer-C₁₄, 1-O-(2′-(.omega.-methoxypolyethyleneglycol)succinoyl)-2-N-myristoyl-sphing osine;
PEG-Cer-C₂₀, 1-O-(2′-(.omega.-methoxypolyethyleneglycol)succinoyl)-2-N-arachidoyl-sphin gosine;
PBS, phosphate-buffered saline;
THF, tetrahydrofuran;
EGTA, ethylenebis(oxyethylenenitrilo)-tetraacetic acid;
SF-DMEM, serum-free DMEM;
NP40, nonylphenoxypolyethoxyethanol,
1,2 dioleoyl-3 dimethylaminopropane (DODAP),
palmitoyl oleoyl phsphatidylcholine (POPC); and
distearoylphosphatidylcholine (DSPC).

The technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entirety as though set forth in full. Standard reference works setting forth the general principles of recombinant DNA technology include Sambrook, J., et al., Molecular Cloning,: A Laboratory Manual, 2d Ed., Cold Spring Harbor Laboratory Press, Planview, N.Y. (1989); McPherson, M. J., Ed., Directed Mutagenesis: A Practical Approach, IRL Press, Oxford (1991); Jones, J., Amino Acid and Peptide Synthesis, Oxford Science Publications, Oxford (1992); Austen, B. M. and Westwood, O. M. R., Protein Targeting and Secretion, IRL Press, Oxford (1991). Any suitable materials and/or methods known to those of skill can be utilized in carrying out the present invention; however, preferred materials and/or methods are described. Materials, reagents and the like to which reference is made in the following description and examples are obtainable from commercial sources, unless otherwise noted. It is believed that one skilled in the art can, based on the description herein, utilize the present invention to its fullest extent. The entire contents of all of the references (including literature references, issued patents, published patent applications, and co-pending patent applications) cited throughout this application are hereby expressly incorporated by reference.

The compositions and methods provided herein include cationic liposomes comprising at least one immunostimulatory nucleic acid, preferably at least one methylated nucleic acid, more preferably at least one methylated oligonucleotide, and most preferably at least one methylated oligodeoxynucleotide. In specific embodiments, the methylated cytosine residue is part of a CpG dinucleotide motif located in said sequence. The CpG comprises a methyl or hydroxymethyl group attached to the carbon-4 position (4-mC) or carbon-5 position (5-mC) of at least one cytosine. In further embodiments, the methylated nucleic acid sequence may alternatively or additionally comprise methyl modifications of the deoxribose or ribose sugar moiety as described in Henry et a/. 2000 J. Pharmacol. Exp. Ther. 292:468, Zhao et al. 1999 Bioorg. Med. Chem Lett. 9:3453, Zhao et al. 2000 Biorg Med. Chem Lett. 10:1051. In a particularly preferred embodiment, the ODN comprises a methylated nucleic acid sequence that has immunostimulatory activity and is designated an immunostimulatory sequence (“ISS”) in non-methylated form.

A “therapeutic antibody” as used herein refers to any synthetic, recombinant, or naturally occurring antibody that provides a beneficial effect in medical treatment of a subject. Particularly preferred are therapeutic antibodies directed to (i.e. capable of binding) a desired target cell surface membrane antigen and thereby opsonizing the target cell for subsequent lysis by immune effector mechanisms. Suitable antibody therapeutics include both monoclonal and polyclonal antibodies directed to tumor-associated antigens and pathogen antigens. Exemplary therapeutic antibodies include an anti-Her2/neu antibody, an anti-CD20 antibody, an anti-CD33 antibody, an anti-CD22 antibody, an anti-EGF-R antibody, an anti-HLA-DR10β antibody, an anti-MUC1 antibody, an anti-T cell receptor antibody, and the like.

A “target cell antigen” as used herein refers to an antigen of interest to which a therapeutic antibody can be directed and an ADCC response can be directed or stimulated. Preferred antigens are surface membrane antigens, and include tumor-associated antigens and pathogen antigens.

A “tumor-associated antigen” as used herein is a molecule or compound (e.g., a protein, peptide, polypeptide, lipid, glycolipid, carbohydrate and/or DNA) associated with a tumor or cancer cell and which is capable of provoking an immune response when expressed on the surface of an antigen presenting cell in the context of an MHC molecule. Tumor-associated antigens include self antigens, as well as other antigens that may not be specifically associated with a cancer but nonetheless enhance an immune response to and/or reduce the growth of a cancer when administered to an animal. In view of the potential risk of autoimmune reactions, the use of self antigens in the subject vaccines may be limited to non-critical tissues such as breast, prostate, testis, melanocytes, etc. More specific embodiments are provided herein.

A “microbial antigen” as used herein is an antigen of a microorganism and includes but is not limited to, infectious virus, infectious bacteria, infectious parasites and infectious fungi. Microbial antigens may be intact microorganisms, and natural isolates, fragments, or derivatives thereof, synthetic compounds which are identical to or similar to naturally-occurring microbial antigens and, preferably, induce an immune response specific for the corresponding microorganism (from which the naturally-occurring microbial antigen originated). In a preferred embodiment, a compound is similar to a naturally-occurring microorganism antigen if it induces an immune response (humoral and/or cellular) to a naturally-occurring microorganism antigen. Compounds or antigens that are similar to a naturally-occurring microorganism antigen are well known to those of ordinary skill in the art. A non-limiting example of a compound that is similar to a naturally-occurring microorganism antigen is a peptide mimic of a polysaccharide antigen. More specific embodiments are provided herein.

“Subject” or “host” as used herein refers to an organism, male or female, having an immune system, preferably an animal, more preferably a vertebrate, even more preferably a mammal, still even more preferably a rodent, and most preferably a human. Further examples of a subject include, but are not limited to, dogs, cats, cows, horses, pigs, sheep, goats, mice, rabbits, and rats. “Patient” as used herein refers to a subject in need of treatment for a medical condition (e.g., disease or disorder) such as cancer or a pathogenic infection.

“In vivo” as used herein refers to an organism, preferably in a mammal, more preferably in a rodent, and most preferably in a human.

“Immunostimulatory,” “immunostimulatory activity” or “stimulating an immune response,” and grammatical equivalents thereof, as used herein refers to inducing, increasing, enhancing, or modulating an immune response, or otherwise providing a beneficial effect with respect to an immune response. Preferably, and in view of the wide variation in in vitro experimental results reported in the prior art, the immunostimulatory activity of a given formulation and nucleic acid sequence is determined in a suitable in vivo assay as described herein.

“Adjuvant” as used herein refers to any substance that can stimulate or enhance the stimulation of immune responses. Some adjuvants can cause activation of a cell of the immune system, for example, an adjuvant can cause an immune cell to produce and secrete cytokines. Examples of adjuvants that can cause activation of a cell of the immune system include, but are not limited to, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”).

“Immune stimulation” or “inducing an immune response” is broadly characterized as a direct or indirect response of an immune system cell or component to an intervention. These responses can be measured in many ways including activation, proliferation or differentiation of immune system cells (B cells, T cells, dendritic cells, APCs, macrophages, NK cells, NKT cells etc.), up-regulated or down-regulated expression of markers, cytokine, interferon, IgM and IgG release in the serum, splenomegaly (including increased spleen cellularity), hyperplasia and mixed cellular infiltrates in various organs. Further, the stimulation or response may be of innate immune system cells, or of the acquired immune system cells (for example, as by a vaccine containing a normally weak antigen). As demonstrated herein, administration of the subject liposomal nucleic acids results in both expansion and activation of NK cells, macrophages and other critical immune effector cells of the innate immune system. In one embodiment, the compositions find use in improving immune effector mechanisms such as ADCC. In a preferred embodiment, the cationic liposomes comprising an immunostimulatory nucleic acid result in a synergistic effect when used in combination with a therapeutic antibody.

The compositions and methods of the invention include a liposome, and more preferably, a cationic liposome, comprising an immunostimulatory nucleic acid. Such liposomes are well known in the art and include, but are not limited to, unilamellar vesicles, multilamellar vesicles, lipid complexes and lipid particles. Liposomes having one lipid-containing membrane are referred to herein as “unilamellar.” Liposomes having multiple lipid-containing membranes are referred to herein as “multilamellar.” “Lipid bilayer” as used herein refers to a lipid-containing membrane having two layers. In preferred embodiments, the liposomes are multilamellar. The immunostimulatory nucleic acid may be either complexed with or encapsulated by the cationic liposome, and most preferably, is fully encapsulated within a cationic lipid particle.

Nucleic Acids

Nucleic acids suitable for use in the compositions and methods of the present invention include, for example, DNA or RNA. Preferably the nucleic acids are oligonucleotides, more preferably oligodeoxynucleotides (ODNs), and most preferably an ODN comprising an ISS (“ISS ODN”). Preferred ISS include, e.g., certain palindromes leading to hairpin secondary structures (see Yamamoto S., et al. (1992) J. Immunol. 148: 4072-4076), or CpG motifs, as well as other known ISS features (such as multi-G domains, see WO 96/11266). In a particularly preferred embodiment, the nucleic acid comprises at least one CpG motif having a methylated cytosine.

“Nucleic acids” as used herein refer to multiple nucleotides (i.e., molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). Nucleic acids may be, for example DNA or RNA. Preferably the nucleic acids are oligoribonucleotides and more preferably ODNs. Nucleic acids may also be polynucleosides, i.e., a polynucleotide minus the phosphate and any other organic base-containing polymer.

In a preferred embodiment, the oligonucleotides are single stranded and in the range of 5-50 nucleotides (“nt”) in length. However, any oligonucleotides may be used including, for example, large double stranded plasmid DNA in the range of 500-50,000 base pairs (“bp”).

Nucleic acids useful in the compositions and methods of the present invention can be obtained from known sources or isolated using methods well known in the art. The nucleic acids can also be prepared by recombinant or synthetic methods which are equally well known in the art. Such nucleic acids can then be encapsulated in lipid particles and the resulting compositions tested for immunostimulatory activity using the methods of the present invention as described herein.

For use in vivo, nucleic acids may be resistant to degradation (e.g., via endo-and exo-nucleases). Secondary structures, such as stem loops, can stabilize nucleic acids against degradation. Alternatively, nucleic acid stabilization can be accomplished via phosphate backbone modifications. A preferred stabilized nucleic acid has at least a partial phosphorothioate modified backbone (PS). Phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann and Peyman, Chem. Rev. 90:544, 1990; Goodchild, Bioconjugate Chem. 1:165, 1990). As demonstrated herein, however, such modifications are not essential to the utility of the methods and compositions of the present invention.

Thus, oligonucleotides useful in the compositions and methods of the present invention may have a modified phosphate backbone such as, e.g., phosphorothioate, methylphosphonate, methylphosphorothioate, phosphorodithioate, and combinations thereof with each other and/or with phosphodiester oligonucleotide. In addition, other modified oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. PO ODN may be preferred where cellular immune responses are desired, while modified ODN such as, e.g., PS ODN may be preferred where humoral responses are desired.

Numerous other chemical modifications to the base, sugar or linkage moieties are also useful. Bases may be methylated or unmethylated. In the preferred embodiments, methyl or hydroxymethyl groups are attached to the carbon-4 position (4-mC) or carbon-5 position (5-mC) of at least one cytosine. The methylated cytosine is preferably located within a CpG motif in the nucleic acid sequence. Alternatively or additionally, the sugar moiety may be modified with a methyl group as described in the art.

Nucleic acid sequences useful in the compositions and methods of the present invention may be complementary to patient/subject mRNA, such as antisense oligonucleotides, or they may be foreign or non-complementary (e.g., the nucleotide sequences do not specifically hybridize to the patient/subject genome). The nucleic acid sequences may be expressed and the resulting expression products may be RNA and/or protein. In addition, such nucleotide sequences may be linked to appropriate promoters and expression elements, and may be contained in an expression vector. As used herein, the term “non-sequence specific” refers to nucleic acid sequences which are non-complementary and which do not encode expression products.

The nucleic acids of the present invention can be synthesized de novo using any of a number of procedures well known in the art. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986,; Garegg et al., Tet. Let. 27:4055-4058, 1986, Gaffney et al., Tet. Let. 29:2619-2622, 1988). These chemistries can be performed by a variety of automated oligonucleotide synthesizers available in the market. Also, CpG dinucleotides can be produced on a large scale in plasmids, (see Sambrook, T., et al., Molecular Cloning: A Laboratory Manual, Cold Spring Harbor laboratory Press, New York, 1989). Such plasmids may also encode other genes to be expressed such as an antigen-encoding gene in the case of a DNA vaccine. Oligonucleotides can be prepared from existing nucleic acid sequences (e.g., genomic or cDNA) using known techniques, such as those employing restriction enzymes, exonucleases or endonucleases.

For administration in vivo, compositions of the present invention, including components of the compositions, e.g., a lipid component or a nucleic acid component, may be associated with a molecule that results in higher affinity binding to target cell (e.g., B-cell, monocytic cell and natural killer (NK) cell) surfaces and/or increased cellular uptake by target cells. The compositions of the present invention, including components of the compositions, can be ionically or covalently associated with desired molecules using techniques which are well known in the art. A variety of coupling or cross-linking agents can be used, e.g., protein A, carbodiimide, and N-succinimidyl-3-(2-pyridyldithio) propionate (SPDP).

The immune stimulating activity of a nucleic acid sequence in an organism can be determined by simple experimentation, for example, by comparing the sequence in question with other immunostimulatory agents, e.g., other adjuvants, or ISS; or by detecting or measuring the immunostimulatory activity of the sequence in question, e.g., by detecting or measuring the activation of host defense mechanisms or the activation of immune system components. Such assays are well known in the art. Also, one of skill in the art would know how to identify the optimal oligonucleotides useful for a particular mammalian species of interest using routine assays described herein and/or known in the art.

Specific nucleic acid sequences of ODNs suitable for use in the compositions and methods of the invention are described in U.S. Patent Appln. 60/379,343, U.S. patent application Ser. No. 09/649,527, Int. Publ. WO 02/069369, Int. Publ. No. WO 01/15726, U.S. Pat. No. 6,406,705, and Raney et al., Journal of Pharmacology and Experimental Therapeutics, 298:1185-1192 (2001), which are all incorporated herein by reference. Exemplary sequences of the ODNs include, but are not limited to, those nucleic acid sequences shown in Table 1. In preferred embodiments, ODNs used in the compositions and methods of the present invention have a phosphodiester (“PO”) backbone or a phosphorothioate (“PS”) backbone, and at least one methylated cytosine residue in the CpG motif.

TABLE 1

| | | |
|---|---|---|
| ODN NAME | ODN SEQ ID NO | ODN SEQUENCE (5′-3′) |

| | | |
|---|---|---|
| ODN 1 (INX-6295) | SEQ ID NO: 2 | 5′-TAACGTTGAGGGGCAT-3 |
| human c-myc | | |
| * ODN 1m (INX-6303) | SEQ ID NO: 4 | 5′-TAAZGTTGAGGGGCAT-3 |
| ODN 2 (INX-1826) | SEQ ID NO: 1 | 5′-TCCATGACGTTCCTGACGTT-3 |
| * ODN 2m (INX-1826m) | SEQ ID NO: 31 | 5′-TCCATGAZGTTCCTGAZGTT-3 |
| ODN 3 (INX-6300) | SEQ ID NO: 3 | 5′-TAAGCATACGGGGTGT-3 |
| ODN 5 (INX-5001) | SEQ ID NO: 5 | 5′-AACGTT-3 |
| ODN 6 (INX-3002) | SEQ ID NO: 6 | 5′-GATGCTGTGTCGGGGTCTCCGGGC-3′ |
| ODN 7 (INX-2006) | SEQ ID NO: 7 | 5′-TCGTCGTTTTGTCGTTTTGTCGTT-3′ |
| ODN 7m (INX-2006m) | SEQ ID NO: 7 | 5′-TZGTZGTTTTGTZGTTTTGTZGTT-3′ |
| ODN 8 (INX-1982) | SEQ ID NO: 8 | 5′-TCCAGGACTTCTCTCAGGTT-3′ |
| ODN 9 (INX-G3139) | SEQ ID NO: 9 | 5′-TCTCCCAGCGTGCGCCAT-3′ |
| ODN 10 (PS-3082) | SEQ ID NO: 10 | 5′-TGCATCCCCCAGGCCACCAT-3 |
| murine Intracellular | | |
| Adhesion Molecule-1 | | |
| ODN 11 (PS-2302) | SEQ ID NO: 11 | 5′-GCCCAAGCTGGCATCCGTCA-3′ |
| human Intracellular | | |
| Adhesion Molecule-1 | | |
| ODN 12 (PS-8997) | SEQ ID NO: 12 | 5′-GCCCAAGCTGGCATCCGTCA-3′ |
| human Intracellular | | |
| Adhesion Molecule-1 | | |
| ODN 13 (US3) | SEQ ID NO: 13 | 5′-GGT GCTCACTGC GGC-3′ |
| human erb-B-2 | | |
| ODN 14 (LR-3280) | SEQ ID NO: 14 | 5′-AACC GTT GAG GGG CAT-3′ |
| human c-myc | | |
| ODN 15 (LR-3001) | SEQ ID NO: 15 | 5′-TAT GCT GTG CCG GGG TCT TCG GGC- |
| human c-myc | | 3′ |
| ODN 16 (Inx-6298) | SEQ ID NO: 16 | 5′-GTGCCG GGGTCTTCGGGC-3′ |
| ODN 17 (hIGF-1R) | SEQ ID NO: 17 | 5′-GGACCCTCCTCCGGAGCC-3′ |
| human Insulin Growth | | |
| Factor 1-Receptor | | |
| ODN 18 (LR-52) | SEQ ID NO: 18 | 5′-TCC TCC GGA GCC AGA CTT-3′ |
| human Insulin Growth | | |
| Factor 1-Receptor | | |
| ODN 19 (hEGFR) | SEQ ID NO: 19 | 5′-AAC GTT GAG GGG CAT-3′ |
| human Epidermal | | |
| Growth Factor- | | |
| Receptor | | |
| ODN 20 (EGFR) | SEQ ID NO: 20 | 5′-CCGTGGTCA TGCTCC-3′ |
| Epidermal Growth | | |
| Factor-Receptor | | |
| ODN 21 (hVEGF) | SEQ ID NO: 21 | 5′-CAG CCTGGCTCACCG CCTTGG-3′ |
| human Vascular | | |
| Endothelial Growth | | |
| Factor | | |
| ODN 22 (PS-4189) | SEQ ID NO: 22 | 5′-CAG CCA TGG TTC CCC CCA AC-3′ |
| murine Phosphokinase | | |
| C-alpha | | |
| ODN 23 (PS-3521) | SEQ ID NO: 23 | 5′-GTT CTC GCT GGT GAG TTT CA-3′ |
| ODN 24 (hBcl-2) | SEQ ID NO: 24 | 5′-TCT CCCAGCGTGCGCCAT-3′ |
| human Bcl-2 | | |
| ODN 25 (hC-Raf-1) | SEQ ID NO: 25 | 5′-GTG CTC CAT TGA TGC-3′ |
| human C-Raf-s | | |
| ODN #26 (hVEGF-R1) | SEQ ID NO: 26 | 5′-GAGUUCUGAUGAGGCCGAAAGGCCG |
| human Vascular | | AAAGUCUG-3′ |
| Endothelial Growth | | |
| Factor Receptor-1 | | |
| ODN #27 | SEQ ID NO: 27 | 5′-RRCGYY-3′ |
| ODN #28 (INX-3280) | SEQ ID NO: 28 | 5′-AACGTTGAGGGGCAT-3′ |
| ODN #29 (INX-6302) | SEQ ID NO: 29 | 5′-CAACGTTATGGGGAGA-3′ |
| ODN #30 (INX-6298) | SEQ ID NO: 30 | 5′-TAACGTTGAGGGGCAT-3′ |
| human c-myc | | |

“Z” represents a methylated cytosine residue.
Note
ODN 14 is a 15-mer oligonucleotide and ODN 1 is the same oligonucleotide having a thymidine added onto the 5′ end making ODN 1 into a 16-mer. No difference in biological activity between ODN 14 and ODN 1 has been detected and both exhibit similar immunostimulatory activity (Mui et al., 2001)

Liposomes

Liposomes and methods for their preparation are well known in the art, and any of number of liposomal formulations may find advantageous use herein, including those described in U.S. Pat. Nos. 6,465,439; 6,379,698; 6,365,611; 6,093,816, and 6,693,086, the disclosures of which are incorporated herein by reference. Preferred liposomes are liposomes comprising cationic lipids, and still more preferably, the cationic lipid particle formulations described herein and more fully described in, for example, U.S. Pat. Nos. 5,785,992; 6,287,591; 6,287,591 B1; co-pending U.S. Patent Appln. Ser. No. 60/379,343, and co-pending U.S. patent application Ser. No. 09/649,527 all incorporated herein by reference.

In a particularly preferred embodiment, the cationic liposome comprises DSPC, DODMA, Chol, and PEG-DMG having a ratio of 20:25:45:10 mol/mol. As used herein, the molar amount of each lipid is given in the same order that the lipid is listed (e.g., the ratio of DSPC to DODMA to Chol to PEG-DMG is 20 DSPC: 25 DODMA: 45 Chol; 10 PEG-DMG or “20:25:45:10”). In alternate embodiments the DSPC may be replaced with POPC, the DODMA replaced with DODAP and the PEG-DMG replaced with PEGCer14 or PEGCer20.

The term “lipid” refers to a group of organic compounds that are esters of fatty acids and are characterized by being insoluble in water but soluble in many organic solvents. They are usually divided in at least three classes: (1) “simple lipids” which include fats and oils as well as waxes; (2) “compound lipids” which include phospholipids and glycolipids; and (3) “derived lipids” such as steroids and compounds derived from lipid manipulations. A wide variety of lipids may be used with the invention, some of which are described below.

The term “charged lipid” refers to a lipid species having either a cationic charge or negative charge or which is a zwitterion which is not net neutrally charged, and generally requires reference to the pH of the solution in which the lipid is found.

Cationic charged lipids at physiological pH include, but are not limited to, N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleyloxy)propyl)-N,N,N-trimethylammonium chloride (“DOTAP”); 3b-(N-(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of catioinic lipids are available which can be used in the present invention. These include, for example, Lipofectin™ (commercially available cationic liposomes comprising DOTMA and 1,2-dioleoyl-sn-3-phosphoethanolamine (“DOPE”), from GIBCO/BRL, Grand Island, N.Y., U.S.A); and Lipofectin™ (commercially available cationic liposomes comprising N-(1-(2,3-dioleyloxy)propyl)-N-(2-(sperminecarboxamido)ethyl)-N,N-dimethylammonium trifluoroacetate (“DOSPA”).

Some cationic charged lipids are titratable, that is to say they have a pKa at or near physiological pH, with the significant consequence for this invention that they are strongly cationic in mild acid conditions and weakly (or not) cationic at physiological pH. Such cationic charged lipids include, but are not limited to, N-(2,3-dioleyloxy)propyl)-N,N-dimethylammonium chloride (“DODMA”) and 1,2-Dioleoyl-3-dimethylammonium-propane (“DODAP”). DMDMA is also a useful titratable cationic lipid.

Anionic charged lipids at physiological pH include, but are not limited to, phosphatidyl inositol, phosphatidyl serine, phosphatidyl glycerol, phosphatidic acid, diphosphatidyl glycerol, poly(ethylene glycol)-phosphatidyl ethanolamine, dimyristoylphosphatidyl glycerol, dioleoylphosphatidyl glycerol, dilauryloylphosphatidyl glycerol, dipalmitoylphosphatidyl glycerol, distearyloylphosphatidyl glycerol, dimyristoyl phosphatic acid, dipalmitoyl phosphatic acid, dimyristoyl phosphatidyl serine, dipalmitoyl phosphatidyl serine, brain phosphatidyl serine, and the like.

Some anionic charged lipids may be titrateable, that is to say they would have a pKa at or near physiological pH, with the significant consequence for this invention that they are strongly anionic in mild base conditions and weakly (or not) anionic at physiological pH. Such anionic charged lipids can be identified by one skilled in the art based on the principles disclosed herein.

The term “neutral lipid” refers to any of a number of lipid species which exist either in an uncharged or neutral zwitterionic form at physiological pH. Such lipids include, for example, diacylphosphatidylcholine, diacylphosphatidylethanolamine, ceramide, sphingomyelin, cephalin, cholesterol, cerebrosides and diacylglycerols.

Certain preferred lipid formulations used in the invention include aggregation preventing compounds such as PEG-lipids or polyamide oligomer-lipids (such as an ATTA-lipid), and other steric-barrier or “stealth”-lipids, detergents, and the like. Such lipids are described in U.S. Pat. No. 4,320,121, U.S. Pat. No. 5,820,873, U.S. Pat. No. 5,885,613, Int. Publ. No. WO 98/51278, and U.S. patent application Ser. No. 09/218,988 relating to polyamide oligomers, all incorporated herein by reference. These lipids and detergent compounds prevent precipitation and aggregation of formulations containing oppositely charged lipids and therapeutic agents. These lipids may also be employed to improve circulation lifetime in vivo (see Klibanov et al. (1990) FEBS Letters, 268 (1): 235-237), or they may be selected to rapidly exchange out of the formulation in vivo (see U.S. Pat. No. 5,885,613, incorporated herein by reference).

A preferred embodiment of the invention employs exchangeable steric-barrier lipids (as described in U.S. Pat. No. 5,820,873, U.S. Pat. No. 5,885,613, and U.S. patent application Ser. No. 09/094540 and U.S. Pat. No. 6,320,017, all incorporated herein by reference). Exchangeable steric-barrier lipids such as PEG2000-CerC14 and ATTA8-CerC14 are steric-barrier lipids which rapidly exchange out of the outer monolayer of a lipid particle upon administration to a subject/patient. Each such lipid has a characteristic rate at which it will exchange out of a particle depending on a variety of factors including acyl chain length, saturation, size of steric barrier moiety, membrane composition and serum composition, etc. Such lipids are useful in preventing aggregation during particle formation, and their accelerated departure from the particle upon administration provides benefits, such as programmable fusogenicity and particle destabilizing activity, as described in the above noted patent submissions.

Some liposomes may employ targeting moieties designed to encourage localization of liposomes at certain target cells or target tissues. Targeting moieties may be associated with the outer bilayer of the lipid particle (i.e., by direct conjugation, hydrophobic interaction or otherwise) during formulation or post-formulation. These methods are well known in the art. In addition, some liposomes may employ fusogenic polymers such as PEAA, hemagluttinin, other lipo-peptides (see U.S. Pat. No. 6,417,326, and U.S. patent application Ser. No. 09/674,191, all incorporated herein by reference) and other features useful for in vivo and/or intracellular delivery.

In another preferred embodiment, the liposomes of the present invention comprise sphingomyelin and cholesterol (“sphingosomes”). In a preferred embodiment, the liposomes used in the compositions and methods of the present invention are comprised of sphingomyelin and cholesterol and have an acidic intraliposomal pH. The liposomes comprising sphingomyelin and cholesterol have several advantages when compared to other formulations. The sphingomyelin/cholesterol combination produces liposomes which have extended circulation lifetimes, are much more stable to acid hydrolysis, have significantly better drug retention characteristics, have better loading characteristics into tumors and the like, and show significantly better anti-tumor efficacy than other liposomal formulations tested.

In a preferred embodiment, the liposomes of the present invention comprise a cationic compound of Formula I and at least one neutral lipid as follows (and fully described in U.S. Pat. No. 5,785,992, incorporated herein by reference).ln a preferred embodiment, the LNA formulations of the present invention comprise a cationic compound of Formula I and at least one neutral lipid as follows (and fully described in U.S. Pat. No. 5,785,992, incorporated herein by reference).

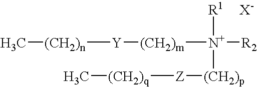

In Formula I, R¹ and R² are each independently C₁ to C₃; alkyl. Y and Z are akyl or alkenyl chains and are each independently: —CH₂CH₂CH₂CH₂CH₂—, —CH═CHCH₂CH₂CH₂—, —CH₂ CH═CHCH₂CH₂—, —CH₂CH₂CH═CHCH₂—, —CH₂CH₂CH₂CH═CH—, —CH═CHCH═CHCH₂—, —CH═CHCH₂CH═CH—, or —CH₂CH═CHCH═CH—, with the proviso that Y and Z are not both —CH₂CH₂CH₂CH₂CH₂—. The letters n and q denote integers of from 3 to 7, while the letters m and p denote integers of from 4 to 9, with the proviso that the sums n+m and q+p are each integers of from 10 to 14. The symbol X⁻ represents a pharmaceutically acceptable anion. In the above formula, the orientation of the double bond can be either cis or trans, however the cis isomers are generally preferred.

In another preferred embodiment, the cationic liposomes are of Formula I, wherein R¹ and R² are methyl and Y and Z are each independently: —CH═CHCH₂CH₂CH₂—, —CH₂CH═CHCH₂CH₂—, —CH₂CH₂CH═CHCH₂— or —CH₂CH₂CH₂CH═CH—. In preferred embodiments, R¹ and R² are methyl; Y and Z are each —CH═CHCH₂CH₂CH₂—; n and q are both 7; and m and p are both 5. In another preferred embodiment, the cationic compound is DODAC (N,N-dioleyl-N,N-dimethylammonium chloride). DODAC is a known in the art and is a compound used extensively as an additive in detergents and shampoos. DODA is also used as a co-lipid in liposomal compositions with other detergents (see, Takahashi, et al., GB 2147243).

The neutral lipids in the cationic liposomes of the present invention can be any of a variety of neutral lipids which are typically used in detergents, or for the formation of micelles or liposomes. Examples of neutral lipids which are useful in the present compositions are, but are not limited to, diacylphosphatidylcholine, diacylphosphatidylethanolamine, ceramide, sphingomyelin, cephalin, cardiolipin, and cerebrosides. In a preferred embodiment, the present compositions will include one or more neutral lipids which are diacylphosphatidylcholine, diacylphosphatidylethanolamine, ceramide or sphingomyelin. The acyl groups in these neutral lipids are preferably acyl groups derived from fatty acids having C₁₀-C₂₄ carbon chains. More preferably the acyl groups are lauroyl, myristoyl, palmitoyl, stearoyl or oleoyl. In particularly preferred embodiments, the neutral lipid will be 1,2-sn-dioleoylphosphatidylethanolamine.

The anion, X—, can similarly be any of a variety a pharmaceutically acceptable anions. These anions can be organic or inorganic, including for example, Br⁻, Cl⁻, F⁻, I⁻, sulfate, phosphate, acetate, nitrate, benzoate, citrate, glutamate, and lactate. In preferred embodiments, X⁻ is Cl⁻ or AcO⁻.

In addition to the other components described herein, the compositions of the present invention may contain a pharmaceutically acceptable carrier. Pharmaceutically acceptable carriers are well known in the art. The choice of carrier is determined in part by the particular composition to be administered as well as by the particular method used to administer the composition. Preferably, the pharmaceutical carrier is in solution, in water or saline.

In the compositions of the present invention, the ratio of cationic compound to neutral lipid is preferably within a range of from about 25:75 (cationic compound:neutral lipid), or preferably to 75:25 (cationic compound:neutral lipid), or preferably about 50:50.

The cationic compounds that are used in the compositions of the present invention can be prepared by methods known to those of skill in the art using standard synthetic reactions (see March, Advanced Organic Chemistry, 4th Ed., Wiley-Interscience, NY, N.Y. (1992), incorporated herein by reference). For example, the synthesis of OSDAC can be carried out by first treating oleylamine with formaldehyde and sodium cyanoborohydride under conditions that result in the reductive alklation of the amine. This approach provides dimethyl oleylamine, which can then be alkylated with stearyl bromide to form the corresponding ammonium salt. Anion exchange results in the formation of OSDAC. Dimethyloleylamine can also be synthesized by treatment of oleyl bromide with a large excess of dimethylamine, and further derivatized as described above.

For cationic compounds in which both fatty acid chains are unsaturated (i.e., DODAC), the following general procedure can be used. An unsaturated acid (i.e., oleic acid) can be converted to its corresponding acyl chloride with such reagents as oxalyl chloride, thionyl chloride, PCI3 or PCI5. The acyl chloride can be treated with an unsaturated amine (i.e., oleylamine) to provide the corresponding amide. Reduction of the amide with, for example, lithium aluminum hydride provides a secondary amine wherein both alkyl groups are unsaturated long chain alkyl groups. The secondary amine can then be treated with alkyl halides such as methyl iodide to provide a quaternary ammonium compound. Anion exchange can then be carried out to provide cationic compounds having the desired pharmaceutically acceptable anion. The alkylamine precursor can be synthesized in a similar manner. For example, treatment of an alkyl halide with a methanolic solution of ammonia in large excess will produce the required amine after purification. Alternatively, an acyl chloride, produced by treatment of the appropriate carboxylic acid with oxalyl chloride, can be reacted with ammonia to produce an amide. Reduction of the amide with LiAlH4 will provide the required alkylamine.

In preferred embodiments, the pharmaceutical compositions of the present invention are formulated as micelles or liposomes. Micelles containing the cationic compounds and neutral lipids of the present invention can be prepared by methods well known in the art. In addition to the micellar formulations of the present compositions, the present invention also provides micellar formulations that include other species such as lysophosphatidylcholine, lysophosphatidylethanolamine, lysophosphatidylserine, lysophosphatidylglycerol, phosphatidylethanolamine-polyoxyethylene conjugate, ceramide-polyoxyethylene conjugate or phosphatidic acid-polyoxyethylene conjugate.

The polyoxyethylene conjugates that are used in the compositions of the present invention can be prepared by combining the conjugating group (i.e. phosphatidic acid or phosphatidylethanolamine) with an appropriately functionalized polyoxyethylene derivative. For example, phosphatidylethanolamine can be combined with omega-methoxypolyethyleneglycol succinate to provide a phosphatidylethanolamine-polyoxyethylene conjugate (see, e.g., Parr, et al., Biochim. Biophys. Acta 1195:21-30 (1994), incorporated herein by reference).

The selection of neutral lipids for use in the compositions and methods of the present invention is generally guided by consideration of, e.g., liposome size and stability of the liposomes in the bloodstream. As described above, the neutral lipid component in the liposomes is a lipid having two acyl groups, (i.e., diacylphosphatidylcholine and diacylphosphatidyl-ethanolamine). Lipids having a variety of acyl chain groups of varying chain length and degree of saturation are available or may be isolated or synthesized by well-known techniques. In general, less saturated lipids are more easily sized, particularly when the liposomes must be sized below about 0.3 microns, for purposes of filter sterilization. In one group of embodiments, lipids containing saturated fatty acids with carbon chain lengths in the range of C14 to C22 are preferred. In another group of embodiments, lipids with mono or diunsaturated fatty acids with carbon chain lengths in the range of C14 to C22 are used. Additionally, lipids having mixtures of saturated and unsaturated fatty acid chains can be used.

Liposomes useful in the compositions and methods of the present invention may also be composed of sphingomyelin or phospholipids with other head groups, such as serine and inositol. Still other liposomes useful in the present invention will include cholesterol, diglycerides, ceramides, phosphatidylethanolamine-polyoxyethylene conjugates, phosphatidic acid-polyoxyethylene conjugates, or polyethylene glycol-ceramide conjugates (e.g., PEG-Cer-C14 or PEG-Cer-C20). Methods used in sizing and filter-sterilizing liposomes are discussed below.

A variety of methods are known in the art for preparing liposomes (see e.g., Szoka et al., Ann. Rev. Biophys. Bioeng. 9:467 (1980), U.S. Pat. Nos. 4,235,871, 4,501,728, 4,837,028, the text Liposomes, Marc J. Ostro, ed., Marcel Dekker, Inc., New York, 1983, Chapter 1, and Hope, et al., Chem. Phys. Lip. 40:89 (1986), all of which are incorporated herein by reference). One known method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture that is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Following liposome preparation, the liposomes may be sized to achieve a desired size range and relatively narrow distribution of liposome sizes. A size range of about 0.2-0.4 microns allows the liposome suspension to be sterilized by filtration through a conventional filter, typically a 0.22 micron filter. The filter sterilization method can be carried out on a high throughput basis if the liposomes have been sized down to about 0.2-0.4 microns.

Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. In both methods, the particle size distribution can be monitored by conventional laser-beam particle size discrimination.

Extrusion of liposomes through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. For use in the present inventions, liposomes having a size of from about 0.05 microns to about 0.15 microns are preferred.

As further described below, the compositions of the present invention can be administered to a subject by any known route of administration. Once adsorbed by cells, the liposomes (including the complexes previously described) can be endocytosed by a portion of the cells, exchange lipids with cell membranes, or fuse with the cells. Transfer or incorporation of the polyanionic portion of the complex can take place via any one of these pathways. In particular, when fusion takes place, the liposomal membrane can be integrated into the cell membrane and the contents of the liposome can combine with the intracellular fluid.

As described below in detail, additional components, which may also be therapeutic compounds, may be added to the liposomes of the present invention to target them to specific cell types. For example, the liposomes can be conjugated to monoclonal antibodies or binding fragments thereof that bind to epitopes present only on specific cell types, such as cancer-related antigens, providing a means for targeting the liposomes following systemic administration. Alternatively, ligands that bind surface receptors of the target cell types may also be bound to the liposomes. Other means for targeting liposomes may also be employed in the present invention.

Following a separation step as may be necessary to remove free drug from the medium containing the liposome, the liposome suspension is brought to a desired concentration in a pharmaceutically acceptable carrier for administration to the patient or host cells. Many pharmaceutically acceptable carriers may be employed in the compositions and methods of the present invention. A variety of aqueous carriers may be used, e.g., water, buffered water, 0.4% saline, 0.3% glycine, and the like, and may include glycoproteins for enhanced stability, such as albumin, lipoprotein, globulin. Generally, normal buffered saline (135-150 mM NaCl) will be employed as the pharmaceutically acceptable carrier, but other suitable carriers will suffice. These compositions may be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride. These compositions may be sterilized techniques referred to above or produced under sterile conditions. The resulting aqueous solutions may be packaged for use or filtered under aseptic conditions and lyophilized, the lyophilized preparation being combined with a sterile aqueous solution prior to administration.

The concentration of liposomes in the carrier may vary. In preferred embodiments, the concentration of liposomes is about 0.1-200 mg/ml. Persons of skill would know how to vary these concentrations to optimize treatment with different liposome components or for particular patients. For example, the concentration may be increased to lower the fluid load associated with treatment.

The cells of a subject are usually exposed to the compositions of the present invention by in vivo or ex vivo administration. In the preferred embodiments described herein, the compositions of the present invention are administered systemically, e.g., intravenously, with intramuscular, subcutaneous and topical administration also contemplated. Alternatively, intranasal or intratracheal administration may be used. Intratracheal administration may be provided as a liquid, preferably as an aerosol. For example, nebulizers may be used to create aerosols of droplets of between 70-100 μm in diameter. It will be understood that droplet size should generally be of greater size than the liposomes.

Multiple administrations to a patient are contemplated. The dosage schedule of the treatments will be determined by the disease and the patient's condition. Standard treatments with therapeutic compounds, including immunostimulatory compositions (e.g., vaccines) that are well known in the art may serve as a guide to treatment with liposomes containing the therapeutic compounds. The duration and schedule of treatments may be varied by methods well known to those of skill, but the increased circulation time and decreased in liposome leakage will generally allow the dosages to be adjusted downward from those previously employed. The dose of liposomes of the present invention may vary depending on the clinical condition and size of the animal or patient receiving treatment. The standard dose of the therapeutic compound when not encapsulated may serve as a guide to the dose of the liposome-encapsulated compound. The dose will typically be constant over the course of treatment, although in some cases the dose may vary. Standard physiological parameters may be assessed during treatment that may be used to alter the dose of the liposomes of the invention.

Antibody Therapeutics

In preferred embodiments of the invention, the cationic liposomes comprising immunostimulatory nucleic acids are administered in combination with an antibody therapeutic directed to a target antigen of interest including, e.g., tumor-associated antigens and pathogen antigens. In a particularly preferred embodiment, the antibody therapeutic is directed to a tumor-associated antigen. The phrase “in combination with” as used herein refers to the simultaneous or sequential administration of the subject agents, either within the same formulation or in separate formulations.

In the embodiments described and exemplified herein, the combination of the subject cationic liposomes with antibody therapeutics provides a synergistic effect in inducing a strong innate immune response, and a strong antibody dependent cellular cytotoxicity response in particular. The synergistic effect results from the dramatic expansion and activation of innate immune effector cells obtained with the subject particles combined with the target-specific opsonization capabilities of the antibodies, which together provide an innate immune response having much greater potency. Thus, the cationic liposomes comprising an immunostimulatory nucleic acid and antibody therapeutic may be administered together in a single formulation, or co-administered to the animal as separate compositions. Moreover, the immune response can be further enhanced by including an additional immune adjuvant, such as a cytokine, either as part of the same formulation or as part of a co-administration protocol, although the inclusion of such an adjuvant is not a requirement for inducing an effective response.

Suitable antibody therapeutics include both monoclonal and polyclonal antibodies directed to tumor-associated antigens and pathogen antigens, and surface membrane antigens in particular. Exemplary antibody therapeutics include include anti-CD20 antibodies such as RITUXAN™, anti-Her2/neu antibodies such as Herceptin™, anti-CD33 antibodies, anti-CD22 antibodies, anti-EGF-R antibodies, anti-HLA-DR10 antibodies, anti-MUC1 antibodies, and the like.

A non-exhaustive list of antibody therapeutics of interest is listed in Table 2 along with the medical indication for which they used. The listed antibody therapeutics may find use in other indications as well.

TABLE 2

| Antibody | | |
|---|---|---|
| Therapeutic | Action | Indication |
| Orthoclone | Anti-CD3 | Allograft rejection |
| OKT3 ™ | | |
| ReoPro ™ | Anti-IIb/Ilia receptor | Prevention of cardiac ischemic |
| | on platelets | complications |
| Rituxan ™ | Anti-CD20 | Non-Hodgkin's lymphoma |
| Simulect ™ | Binds to T-cells | Organ rejection prophylaxis |
| Remicade ™ | Anti-tumor necrosis | Rheumatoid arthritis, |
| | factor alpha | Crohn's disease |
| | (TNF-a) | |
| Zenapax ™ | Anti-II-2 | Organ rejection prophylaxis |
| Synagis ™ | Anti-RSV (F protein) | Respiratory syncytial virus (RSV) |
| Herceptin ™ | Anti-Her-2 | Metastatic breast cancer |
| Mylotarg ™ | Anti-CD33 | Acute myeloid leukemia |
| Campath ™ | Anti-CD52 | Chronic lymphocytic leukemia |
| Zevalin ™ | Anti-CD20 | Non-Hodgkin's Lymphoma |
| | | (relapsed or refractory low-grade, |
| | | follicular, or transformed B cell) |
| Humira ™ | Anti-tumor necrosis | Rheumatoid arthritis |
| | factor alpha | |
| | (TNF-alpha). | |
| Xolair ™ | Anti-immunoglobulin | Moderate to severe persistent |
| | E (IgE) | asthma |
| Bexxar ™ | Anti-CD20 | CD20 positive, follicular, Non- |
| | | Hodgkin's Lymphoma (NHL) |
| Raptiva ™ | Blocks activation | Chronic moderate-to-severe |
| | T cells | psoriasis |
| Erbitux ™ | Anti-epidermal | Colorectal cancer |
| | growth factor | |
| | receptor (EGFR) | |
| Avastin ™ | Anti-VEGF | Colorectal cancer |

In the examples below, the subject cationic liposome compositions are combined with antibody therapeutics for improved potency and synergistic therapeutic effects.

Examples of antigens suitable for use in the present invention include, but are not limited to, polypeptide antigens and DNA antigens. Specific examples of antigens are Hepatitis A, Hepatitis B, small pox, polio, anthrax, influenza, typhus, tetanus, measles, rotavirus, diphtheria, pertussis, tuberculosis, and rubella antigens. In a preferred embodiment, the antigen is a Hepatitis B recombinant antigen. In other aspects, the antigen is a Hepatitis A recombinant antigen. In another aspect, the antigen is a tumor antigen. Examples of such tumor-associated antigens are MUC-1, EBV antigen and antigens associated with Burkitt's lymphoma. In a further aspect, the antigen is a tyrosinase-related protein tumor antigen recombinant antigen. Those of skill in the art will know of other antigens suitable for use in the present invention.

Tumor-associated antigens suitable for use in the subject invention include both mutated and non-mutated molecules that may be indicative of single tumor type, shared among several types of tumors, and/or exclusively expressed or overexpressed in tumor cells in comparison with normal cells. In addition to proteins and glycoproteins, tumor-specific patterns of expression of carbohydrates, gangliosides, glycolipids and mucins have also been documented. Moingeon, supra. Exemplary tumor-associated antigens for use in the subject cancer vaccines include protein products of oncogenes, tumor suppressor genes and other genes with mutations or rearrangements unique to tumor cells, reactivated embryonic gene products, oncofetal antigens, tissue-specific (but not tumor-specific) differentiation antigens, growth factor receptors, cell surface carbohydrate residues, foreign viral proteins and a number of other self proteins.

Specific embodiments of tumor-associated antigens include, e.g., mutated antigens such as the protein products of the Ras p21 protooncogenes, tumor suppressor p53 and BCR-abl oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. Also included as tumor-associated antigens herein are whole cell and tumor cell lysates as well as immunogenic portions thereof, as well as immunoglobulin idiotypes expressed on monoclonal proliferations of B lymphocytes for use against B cell lymphomas.

Pathogens include, but are not limited to, infectious virus that infect mammals, and more particularly humans. Examples of infectious virus include, but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Also, gram negative and gram positive bacteria serve as antigens in vertebrate animals. Such gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacterpyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcusfaecalis, Streptococcus bovis, Streptococcus (anaerobic sps.), Streptococcus pneumoniae, pathogenic Campylobacter sp., Enterococcus sp., Haemophilus infuenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Additional examples of pathogens include, but are not limited to, infectious fungi that infect mammals, and more particularly humans. Examples of infectious fingi include, but are not limited to: Cryptococcus neoformans, Histoplasma capsulatum, Coccidioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e. protists) include Toxoplasma gondii.

Other Drug Components

Some preferred embodiments of the invention further comprise other therapeutic agents, e.g., drugs or bioactive agents. These additional components may provide direct additional therapeutic benefit or additional immune-stimulating benefits. A wide variety of therapeutic compounds may be delivered by the compositions and methods of the present invention. Examples of therapeutic compounds include, but are not limited to, nucleic acids, proteins, peptides, oncolytics, anti-infectives, anxiolytics, psychotropics, immunomodulators, ionotropes, toxins such as gelonin and inhibitors of eucaryotic protein synthesis, and the like. Preferred therapeutic compounds for entrapment in the lipid component of the present invention are those which are lipophilic cations. Among these are therapeutic agents of the class of lipophilic molecules which are able to partition into a lipid bilayer phase of a liposome, and which therefore are able to associate with the liposomes in a membrane form. Further examples of therapeutic compounds include, but are not limited to, prostaglandins, amphotericin B, methotrexate, cisplatin and derivatives, progesterone, testosterone, estradiol, doxorubicin, epirubicin, beclomethasone and esters, vitamin E, cortisone, dexamethasone and esters, betamethasone valerete and other steroids, the fluorinated quinolone antibacterial ciprofloxacin and its derivatives, and alkaloid compounds and their derivatives. Among the alkaloid derivatives are swainsonine and members of the vinca alkaloids and their semisynthetic derivatives, such as, e.g., vinblastine, vincristine, vindesin, etoposide, etoposide phosphate, and teniposide. Among this group, vinblastine and vincristine, and swainsonine are particularly preferred. Swainsonine (Creaven and Mihich, Semin. Oncol. 4:147 (1977) has the capacity to stimulate bone marrow proliferation (White and Olden, Cancer Commun. 3:83 (1991)). Swainsonine also stimulates the production of multiple cytokines including IL-1, IL-2, TNF, GM-CSF and interferons (Newton, Cancer Commun. 1:373 (1989); Olden, K., J. Natl. Cancer Inst., 83:1149 (1991)). Further Swainsonine reportedly induces B- and T-cell immunity, natural killer T-cell and macrophage-induced destruction of tumor cells in vitro, and when combined with interferon, has direct anti-tumor activity against colon cancer and melanoma cancers in vivo (Dennis, J., Cancer Res., 50:1867 (1990); Olden, K., Pharm. Ther. 44:85 (1989); White and Olden, Anticancer Res., 10:1515 (1990)). Other alkaloids useful in the compositions and methods of the present invention include, but are not limited to, paclitaxel (taxol) and synthetic derivatives thereof. Additional drug components include, but are not limited to, any bioactive agents known in the art that can be incorporated into lipid particles.

These additional drug components may be encapsulated by or otherwise associated with the cationic liposomes described herein. Alternatively, the compositions of the invention may include drugs or bioactive agents that are not associated with the cationic liposome, including the therapeutic antibodies. Such drugs or bioactive agents may be in separate liposomes or co-administered as described herein.

Kits

The compositions of the invention can be provided as kits. In one embodiment, the kit comprises a cationic liposome comprising an immunostimulatory nucleic acid. In a preferred embodiment, the immunostimulatory nucleic acid comprises at least one CpG dinucleotide having a methylated cytosine. In further preferred embodiments, the kit comprises a cationic liposome comprising an immunostimulatory nucleic acid and a therapeutic antibody. In a preferred embodiment, the kit comprises a cationic liposome comprising an immunostimulatory nucleic acid in one vial and a therapeutic antibody in a separate vial. In a further preferred embodiment, the kit comprises a cationic liposome comprising an immunostimulatory nucleic acid and a therapeutic antibody present in the same vial.

Manufacturing of Compositions

Manufacturing the compositions of the invention may be accomplished by any technique, but most preferred are the ethanol dialysis or detergent dialysis methods detailed in the following publications, patents, and applications each incorporated herein by reference: U.S. Pat. No. 5,705,385; U.S. Pat. No. 5,976,567; U.S. patent application Ser. No. 09/140,476; U.S. Pat. No. 5,981,501; U.S. Pat. No. 6,287,591; Int. Publ. No. WO 96/40964; and Int. Publ. No. WO 98/51278. These manufacturing methods provide for small and large scale manufacturing of immunostimulatory compositions comprising therapeutic agents encapsulated in a lipid particle, preferably lipid-nucleic acid particles. The methods also generate such particles with excellent pharmaceutical characteristics.

Additional components such as antigens or cytotoxic agents may be added to the cationic liposomes of the present invention using any number of means well known in the art including, e.g. 1) passive encapsulation during the formulation process (e.g., the component can be added to the solution containing the ODN); 2) addition of glycolipids and other antigenic lipids to an ethanol lipid mixture and formulated using the ethanol-based protocols described herein; 3) insertion into the lipid vesicle (e.g., antigen-lipid can be added into formed lipid vesicles by incubating the vesicles with antigen-lipid micelles); and 4) the antigen or other component can be added post-formulation (e.g., coupling in which a lipid with a linker moiety is included into formulated particle, and the linker is activated post formulation to couple a desired antigen). Standard coupling and cross-linking methodologies are well known in the art. An alternative preparation incorporates the antigen into a cationic liposome that does not contain a nucleic acid, and these liposomes are mixed with liposomal nucleic acids prior to administration to the subject.

### CHARACTERIZATION OF COMPOSITIONS USED IN THE METHODS OF THE PRESENT INVENTION

Preferred characteristics of the liposomes used in the compositions and methods of the present invention are as follows.

The preferred liposomes of the invention comprise a lipid membrane (generally a phospholipid bilayer) exterior that fully encapsulates an interior space. These liposomes, also sometimes herein called lipid membrane vesicles, are small particles with mean diameter 50-200 nm, preferably 60-130 nm. Most preferred for intravenous administrations are particles of a relatively uniform size wherein 95% of particles are within 30 nm of the mean. The nucleic acid and other bioactive agents are contained in the interior space, or associated with an interior surface of the encapsulating membrane.

“Fully encapsulated” as used herein indicates that the nucleic acid in the liposomes is not significantly degraded after exposure to serum or a nuclease assay that would significantly degrade free DNA. In a fully encapsulated system, preferably less than 25% of particle nucleic acid is degraded in a treatment that would normally degrade 100% of free nucleic acid, more preferably less than 10% and most preferably less than 5% of the particle nucleic acid is degraded. Alternatively, full encapsulation may be determined by an OligreenTm assay. Fully encapsulated also suggests that the particles are serum stable, that is, that they do not rapidly decompose into their component parts upon in vivo administration.

These characteristics of the compositions of the present invention distinguish the preferred particles of the invention from lipid-nucleic acid aggregates (also known as cationic complexes or lipoplexes) such as DOTMA/DOPE (LIPOFECTIN™) formulations. These complexes/aggregates are generally much larger (>250 nm) diameter, they do not competently withstand nuclease digestion. They generally decompose upon in vivo administration. These types of cationic lipid-nucleic acid complexes may provide suitable liposome compositions for local and regional applications, such as intramuscular, intra-peritoneal and intrathecal administrations, and more preferably intranasal administration.

The lipid components of the invention can be formulated at a wide range of drug:lipid ratios. “Drug to lipid ratio” as used herein refers to the amount of therapeutic nucleic acid (i.e., the amount of nucleic acid which is encapsulated and which will not be rapidly degraded upon exposure to the blood) in a defined volume of preparation divided by the amount of lipid in the same volume. This may be determined on a mole per mole basis or on a weight per weight basis, or on a weight per mole basis. Drug to lipid ratio may determine the lipid dose that is associated with a given dose of nucleic acid. In a preferred embodiment, the compositions of the present invention have a drug:lipid ratio in the range of about 0.01 to 0.25 (wt/wt).

Indications, Administration and Dosages

The compositions and methods of the present invention are indicated for use in any patient or organism having a need for immune system stimulation. Such a need encompasses, but is not limited to, most medical fields, such as oncology, inflammation, arthritis & rheumatology, immuno-deficiency disorders. One skilled in the art can select appropriate indications to test for efficacy based on the disclosure herein. In a preferred embodiment, the compositions and methods of the invention are used to treat a neoplasia (any neoplastic cell growth which is pathological or potentially pathological) such as the neoplasia described in the Examples below.

Administration of the compositions of the invention to a subject may be by any method including in vivo or ex vivo methods. In vivo methods can include local, regional or systemic applications. In a preferred embodiment, the compositions are administered intravenously such that particles are accessible to B cells, macrophages or a splenocytes in a patient, and/or the particle can stimulate lymphocyte proliferation, resulting in secretion of IL-6, IL-12, IFNg and/or IgM in said patient. The compositions may be administered as a single formulation where each of the component parts are mixed together. Embodiments of this aspect of the invention include simultaneous administration of a cationic liposome comprising an immunostimulatory nucleic acid with a therapeutic antibody.

Alternatively, the components of the formulation may be co-administered. As used herein, “coadministered” means to administer the cationic liposome and the therapeutic antibody within a time period short enough to provide the enhanced ADCC response demonstrated herein. Generally, the cationic liposome having the immunostimulatory nucleic acid will be administered prior to the therapeutic antibody to enable mobilization and activation of innate immune effector cells prior to opsonization of the target cell by the antibodies. Typical time periods to provide the immunostimulatory benefits of the combined components by coadministering them separately are within one to seven days, within 12 to 72 hours, more preferably within 48 hours, and most preferably within 24 to 48 hours. Preferred embodiments of this aspect of the invention include administration of a cationic liposome comprising an immunostimulatory nucleic acid prior to administration of the therapeutic antibody. Alternatively, the cationic liposome compositions may be administered subsequent to the administration of the therapeutic antibody, depending on the in vivo half-life of the antibody. Antibodies having a suitable half-life of, e.g., two to five days, may be administered prior to the administration of the cationic liposomes.

As demonstrated in the in vivo studies described herein, the tumor status of the animal does not affect their ability to respond to immune stimulation by cationic liposomes in combination with a therapeutic antibody, with blood or spleen immune cells from tumor-free and tumor-bearing animals responding similarly. The in vivo response to these compositions does appear to be dependent on both dose level and dosing regimen. In vivo dosing with increasing concentrations of liposomal nucleic acid formulations from 5 mg/kg to 40 mg/kg resulted in progressively elevated ADCC activity in peripheral blood NK cells. In terms of dosing regimen, a single dose resulted in elevated NK and ADCC activity over 5-7 days, peaking 24-48 hours after injection. Interestingly, multiple administrations were not beneficial: administration of multiple doses within 3-4 days did not result in any enhancement of either NK or ADCC activity compared to a single dose. However, administration of doses at more protracted times, e.g., once or twice per week, did provide some additional benefit.

The compositions of the present invention may be administered by any known route of administration. In one embodiment, the compositions of the present invention are administered via intravenous injection. In another embodiment, intramuscular or subcutaneous injection is employed and in this manner larger-sized (150-300 nm) liposomes can be used. Consequently, the need for costly extrusion steps can be reduced or eliminated, and since the liposomes do not need to circulate, the selection of liposome components can be biased in favor of less expensive materials. For example, the amount of Chol can be reduced, DSPC can be replaced with something less rigid (e.g., POPC or DMPC), and PEG-lipids can be replaced with less expensive PEG-acyl chains. In a still further embodiment, the compositions of the present invention are administered via the respiratory tract, e.g., by intratracheal instillation or intranasal inhalation.

One skilled in the art would know how to identify possible toxicities of formulations, for example, complement activation, coagulation, renal toxicities, liver enzyme assays, etc. Such toxicities may differ between organisms.

Pharmaceutical preparations of compositions usually employ additional carriers to improve or assist the delivery modality. Typically, compositions of the invention will be administered in a physiologically-acceptable carrier such as normal saline or phosphate buffer selected in accordance with standard pharmaceutical practice. Other suitable carriers include water, 0.9% saline, 0.3% glycine, and the like, including glycoproteins for enhanced stability, such as albumin, lipoprotein, globulin, etc.

Dosages of cationic liposomes depend on the desired lipid dosage, the desired nucleic acid dosage, and the drug:lipid ratio of the composition. One skilled in the art can select proper dosages based on the information provided herein. Similarly, immunotherapy protocols for the therapeutic antibodies contemplated for use herein are well known in the art and/or readily ascertainable by the skilled artisan.

In particular, one skilled in the art would know how to calculate dosage amounts for a subject, particularly a mammal, and more particularly a human, based on the dosage amounts described herein. Specific conversion factors for converting dosage amounts from one animal to another (e.g., from mouse to human) are well known in the art and are fully described, e.g., on the Food and Drug Administration Web site at: www.fda.gov/cder/cancer/animalframe.htm (in the oncology tools section), incorporated herein by reference. As compared to known immunostimulatory compositions having free nucleic acids, the immunostimulatory compositions and methods of the present invention may utilize reduced amounts of nucleic acids to stimulate enhanced immune responses in vivo. Moreover, as demonstrated herein, the synergistic combination of cationic liposomes and antibodies also enables smaller amounts of antibodies to be used while still maintaining superior efficacy.

The amount of nucleic acids in the formulations of the present invention will generally vary between about 0.001-60 mg/kg (mg nucleic acids per kg body weight of a mouse per dose). In preferred embodiments for intravenous (i.v.) administration, the compositions and methods of the present invention utilize about 1-50 mg/kg, more preferably about 5-20 mg/kg. In preferred embodiments for subcutaneous (s.c.) administration, the compositions and methods of the present invention utilize about 1-10 mg/kg, and more preferably about 1-5 mg/kg, usually about about 3-5 mg/kg. The amount of antigen associated with the lipid particles of the present invention is preferably about 0.04-40 mg/kg, and more preferably about 0.04-4 mg/kg. As described above, one skilled in the art could readily determine suitable dosage amounts for other mammals given the dosage amounts described herein, based on the well-known conversion factors identified above and further empirical testing.

The formulations of the invention may be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the immunostimulatory compositions of the present invention can be administered to a subject by any mode allowing uptake by the appropriate target cells. “Administering” the immunostimulatory composition of the present invention may be accomplished by any means known to the skilled artisan. Preferred routes of administration include but are not limited to parenteral injection (subcutaneous, intradermal, intravenous, parenteral, intraperitoneal, intrathecal, etc.), as well as mucosal, intranasal, intratracheal, inhalation, and intrarectal, intravaginal; or oral, transdermal (e.g., via a patch). An injection may be in a bolus or a continuous infusion.

For example, the compositions of the present invention can be administered by intramuscular or intradermal injection, or other parenteral means, or by biolistic “gene-gun” application to the epidermis. The compositions of the present invention may also be administered, for example, by inhalation, topically, intravenously, orally, implantation, rectally, or vaginally. Suitable liquid or solid pharmaceutical preparation forms are, for example, aqueous or saline solutions for injection or inhalation, encochleated, coated onto microscopic gold particles, and nebulized. For a brief review of present methods for drug delivery, see Langer, Science 249:1527-1533, 1990, which is incorporated herein by reference.

The pharmaceutical compositions are preferably prepared and administered in dose units. Liquid dose units are vials or ampoules for injection or other parenteral administration. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of several days or weeks apart may be advantageous for boosting the innate immune responses, as described herein.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

In preferred embodiments, the composiitions of the present invention are optionally included in a pharmaceutically-acceptable carrier. “Pharmaceutically-acceptable carrier” as used herein refers to one or more compatible solid or liquid filler, dilutants or encapsulating substances which are suitable for administration to a human or other mammal. “Carrier” as used herein refers to an organic or inorganic ingredient, natural or synthetic, with which the active ingredient is combined to facilitate the application. The components of the immunostimulatory compositions of the present invention also are capable of being comingled with the compounds of the present invention, and with each other, in a manner such that there is no interaction which would substantially impair the desired pharmaceutical efficiency.

Compositions suitable for parenteral administration conveniently comprise sterile aqueous preparations, which can be isotonic with the blood of the recipient. Among the acceptable vehicles and solvents are water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for subcutaneous, intramuscular, intraperitoneal, intravenous, etc. administrations may be found in Remington's Pharmaceutical Sciences, Mack Publishing Company, Easton, Pa.

A variety of administration routes are available. The particular mode selected will depend, of course, upon the particular adjuvants or antigen selected, the age and general health status of the subject, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

The compositions may conveniently be presented in unit dosage form and may be prepared by any of the methods well known in the art of pharmacy. All methods include the step of bringing the compounds into association with a carrier that constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within amatrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Experimental

Materials & Methods

The lipid components and nucleic acid components used in the following experiments have been described herein. The preferred embodiment of the cationic liposome used in the following examples is a cationic liposome is composed of POPC:CH:DODMA:PEG-DMG at a 20:45:25:10 ratio.

All ODNs used in the following experiments have a phosphodiester backbone unless otherwise noted. The term “free” as used herein refers to an ODN not present in a lipid-nucleic acid composition.

Plasmid DNA employed was the luciferase expression plasmid, pCMVIuc18, (also called pCMVLuc). Plasmid was produced in E. Coli, isolated and purified as described previously (Wheeler, J. J., Palmer, L., Ossanlou, M., MacLachlan, I., Graham, R. W., Zhang, Y. P., Hope, M. J., Scherrer, P., & Cullis, P. R. (1999) Gene Ther. 6, 271-281.). (See also Mortimer I, Tam P, MacLachlan I, Graham R W, Saravolac E G, Joshi P B. Cationic lipid mediated transfection of cells in culture requires mitotic activity. Gene Ther. 1999; 6: 403-411.).

Phosphodiester (PO) and phosphorothioate (PS) ODN were purchased from Hybridon Specialty Products (Milford, Mass.) or were synthesized at Inex Pharmaceuticals (Burnaby, BC, Canada). Methylated ODN were manufactured by standard techniques at Inex Pharmacueticals (USA), Inc. (Hayward, Calif.). The backbone composition was confirmed by ³¹P-NMR. All ODN were specifically analyzed for endotoxin and contained less than 0.05 EU/mg.

### EXAMPLE 1

This series of experiments was designed to investigate the ability of cationic liposomes comprising an immunostimulatory nucleic acid to mediate ADCC and activate NK and LAK.

Materials and Methods

Mice. In this experiment, 60 C3H female mice, 8-9 weeks old (22-25 g) by the time of th experiment were used. The animals were housed in groups of four.

Dosages. There were three treatment groups plus a control, 5 time points. Administrations of test samples and controls were via intravenous tail vein injections with injection volume dependent on body weight (e.g., 200 ml for a 20 g mouse, 350 ml for a 25 g mouse, etc.). Animals will receive 20 mg ODN/kg dose of ODN 2 [SEQ ID NO:1] PS free or ODN 1m [SEQ ID NO:4] free or cationic liposome comprising ODN 1m [SEQ ID NO:4] per injection; at 2 mg/ml in PBS.

Harvest. Mice spleen and blood were harvested (no sterile conditions required). Tissues were dissociated and cells collected for in vitro analysis.

Data Analysis. Blood and splenic cells will be used in CTL/ADCC assays against P815, YAC-1, Daudi target cells in the presence and absence of anti-CD20 Ab (Rituxan™ and/or mouse-anti-human IgG1) and SKBR-3 target cells in the presence and absence of Herceptin™; and analysed on FACS for NK and Monocytes/Macrophages activation (DX5/CD16-CD69, CD11b/CD16-CD69, Mac-3/CD16-CD69).

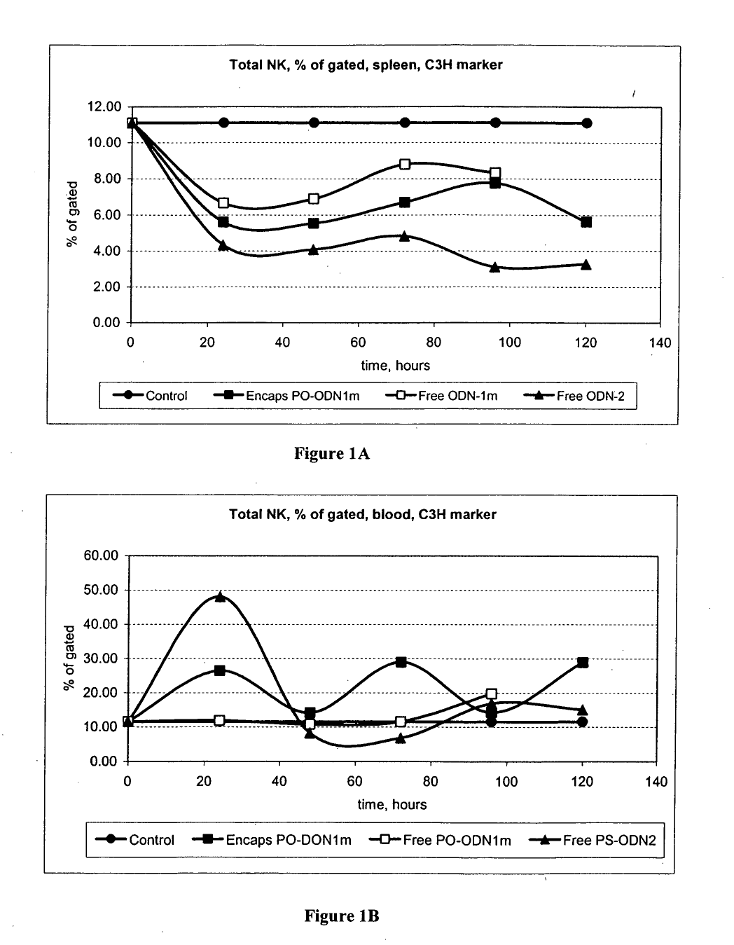

Results. Possible effector cells expansion/migration following single injection of ODN 2 [SEQ ID NO: 1] PS free, ODN 1m [SEQ ID NO:4] free or cationic liposome comprising ODN 1m [SEQ ID NO:4]. All three formulations caused sharp decrease in total NK population in spleen by day 1, and this decrease persisted with little change through days 1-5, with highest effect from ODN 2 [SEQ ID NO:1] PS free (to 4% down from control level of 11%), followed by cationic liposome comprising ODN 1m [SEQ ID NO:4] (to 6%), and the lowest impact from ODN 1m [SEQ ID NO:4] free (to 7%) (FIG. 1A). At the same time, ODN 1m [SEQ ID NO:4] free had no effect on blood NK population; ODN 2 [SEQ ID NO:1] PS free caused increase of NK in blood at day 1 (50%) which returned to control levels (˜10%) by days 2-5; effect of cationic liposome comprising ODN 1m [SEQ ID NO:4] on NK blood population appears to be cyclical, with highest levels at ˜30% (FIG. 1B).

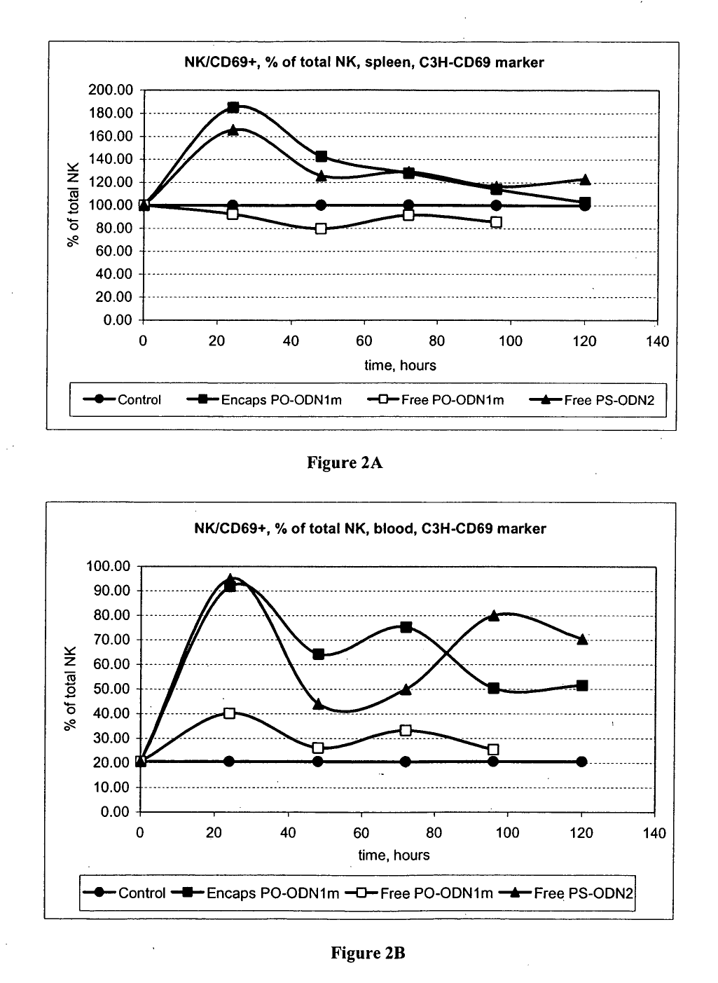

Effector cells activation following single injection of ODN 2 [SEQ ID NO: 1] PS free, ODN 1m [SEQ ID NO:4] free or cationic liposome comprising ODN 1m [SEQ ID NO:4] (CD69 expression). Level of expression of CD69 was assessed by % of CD69-expressing cells of particular cells' population. Although total number of NK cells in spleen decreases upon ODN injection (FIG. 2A), the % of CD69+ NK cells of total NK population increases (FIG. 2B): ODN 2 [SEQ ID NO:1] PS free and cationic liposome comprising ODN 1m [SEQ ID NO:4] cause similar levels of activation (up to 80-90% from 50% of control group at day 1, followed by decrease to 60% at days 2-5).

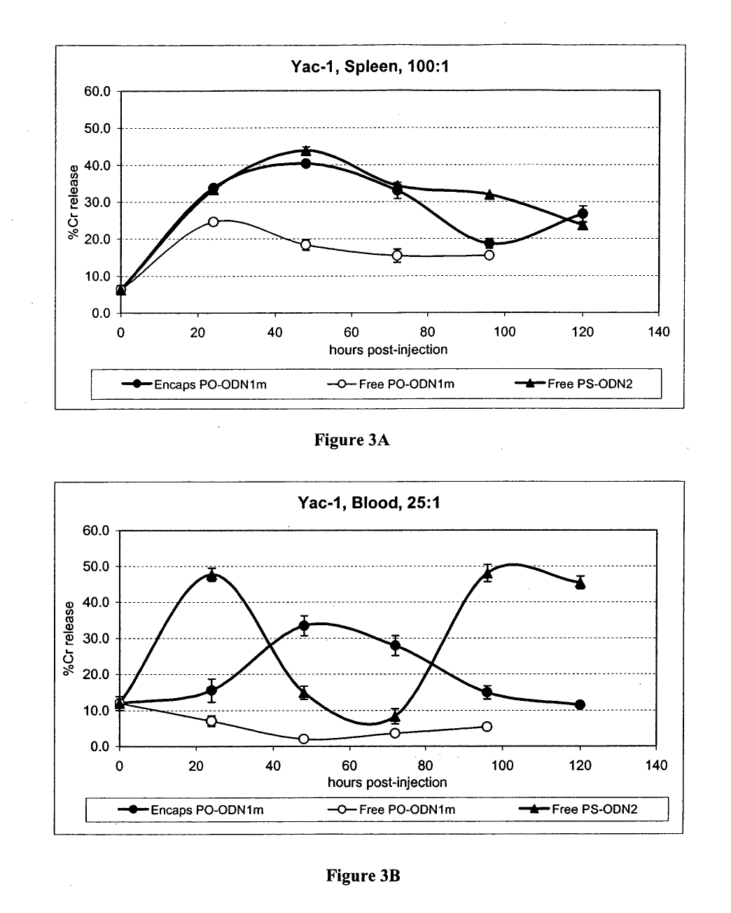

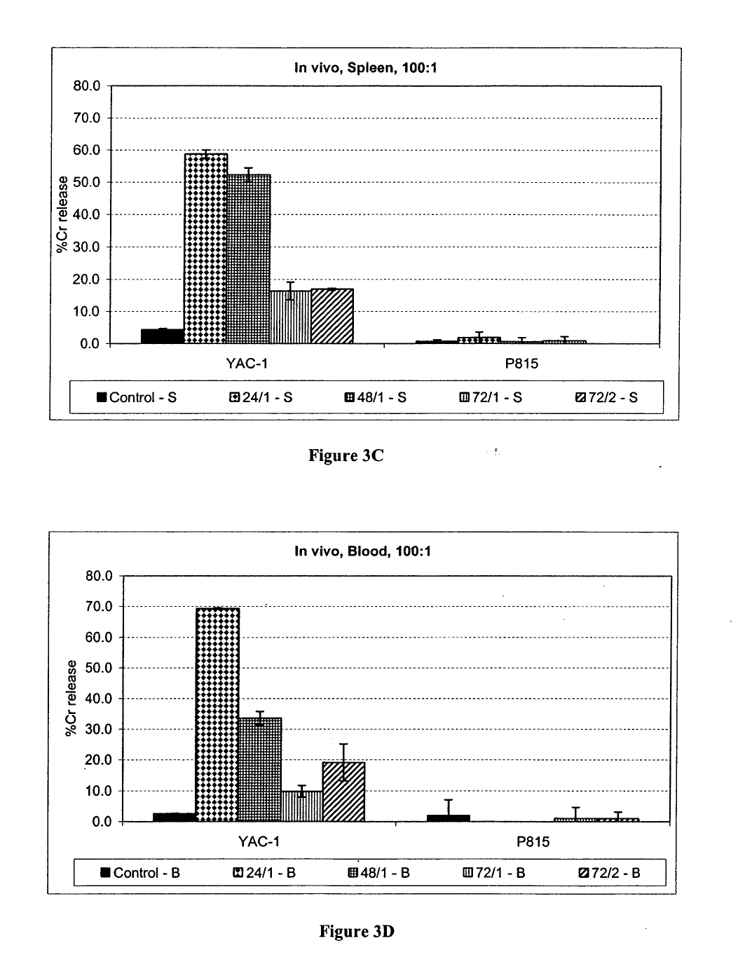

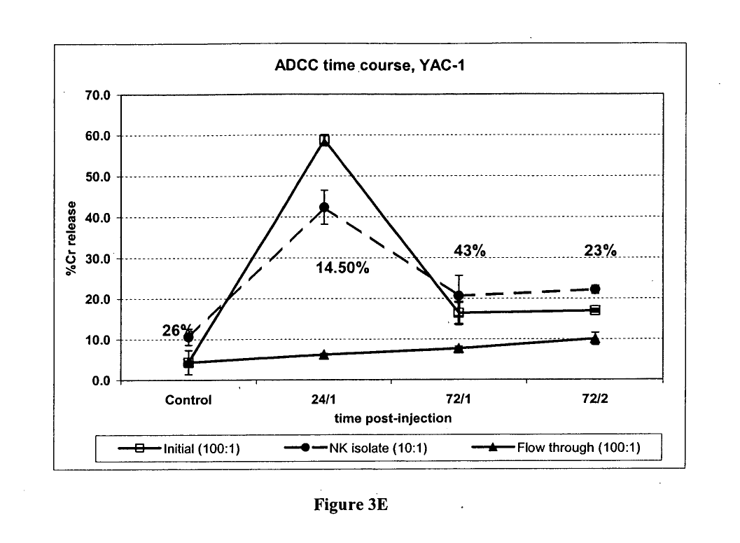

NK activation as measured by killing Yac-1 target in 4-hour Cr assay. IV administration of free ODN 1m [SEQ ID NO:4] causes peak activity in spleen cells at 24 hours post-injection (25% Cr release) with consequent decline to 15-20% levels at days 2-4 (FIG. 3A). A cationic liposome comprising ODN 1m [SEQ ID NO:4] elevates Cr release to 35-40% and prolongs the elevation to 24-72 hours post-injection, with decline to the level of free ODN by day 4. Free ODN 2 [SEQ ID NO:1] PS exhibited the same level of Cr release as cationic liposome comprising ODN 1m [SEQ ID NO:4] at days 1-3, with more gradual decline at days 4-5. In blood (FIG. 3B), free ODN 1m [SEQ ID NO:4] did not stimulate activity against Yac-1 target, and stimulation by cationic liposomes comprising ODN 1m [SEQ ID NO:4] peaks at days 2-3 (28-33% Cr release), restoring to control levels by days 4-5. ODN 2 [SEQ ID NO:1] PS free demonstrated very different profile of stimulation, with the first peak on day 1, return to control levels at days 2-3, and second peak at days 4-5.

### EXAMPLE 2

This series of experiments was designed to investigate the ability of cationic liposomes comprising an immunostimulatory nucleic acid to mediate ADCC and activate NK and LAK.

Mice. 40 C3H female mice, 6-8 weeks old (20-22 g) by the time of the experiment. The animals were housed in groups of 3 and 4.

Dosages. Two treatment groups plus a control, at 5 time points. Administrations of test samples and controls were via intravenous tail vein injections with injection volume dependent on body weight (e.g., 200 ml for a 20 g mouse, 350 ml for a 25 g mouse, etc.). Animals will receive 20 mg ODN/kg dose of cationic liposomes comprising ODN 1m [SEQ ID NO:4] prepared at 2 mg/ml in PBS.

Harvest. Mice blood, liver, lymph node, and spleen were harvested under sterile conditions. Tissues were dissociated and cells collected for in vitro analysis.

Formulations. Cationic liposomes were made using the pre-formed vesicle (PFV) technique, and utilized EtOH. The reformulated PFV was extruded through a 200 nm filter atop a 100 nm filter for two passes.

Data Analysis. In Group A (in vitro stimulation, groups 6a and b): Splenocytes (control and stimulated with cytokines or ODN) will be analyzed by flow cytometry for phenotype and activation (DX-5/CD16 or CD69 and CD11b/CD16 or CD69 and Mac-3/CD16 or CD69) and activity by 51Cr-release against P815, YAC-1 and (Daudi/MCF-7/SK-BR-3) target cells in the presence and absence of anti-CD20 ab (Rituxan™/Herceptin™/m-a-CD20) on days 0, 1, 2, 3.

In Group B (in vivo stimulation, groups 1-5): Blood, splenic, lymph node or liver cells will be used in ADCC assay against P815, YAC-1 and Daudi target cells in the presence and absence of anti-CD20 ab (mouse-anti-human, IgG1), and analyzsed on FACS for NK and Monocytes/Macrophages activation (DX5/CD16-CD69, CD11b/CD16-CD69, Mac-s/CD16-CD69).

Results. IV administration of cationic liposomes comprising ODN 1m [SEQ ID NO: 4] induces NK cells activation, reflected in increased Cr release from Yac-1 cells, but there is no increase in Cr release from P815 target (FIGS. 3C and D): In the YAC-1 cells, activation of cells upon single injection has similar pattern for all four organs: significant elevation at 24-48 hours followed by decrease at 72 hours (spleen, liver) or gradual decrease at 48 and 72 hours (LN, blood). Although significantly reduced by 72 hours, effectors' activity is still higher than the one of control group. Second injection did not appear beneficial for cells isolated from spleen and LN, and produced slight increase of activity for liver and blood cells.

Major effector population for Yac-1 killing appears to be NK cells (FIG. 3E): In order to identify the effector population with highest impact on ADCC, splenocytes from control and several time points were run through NK cells isolation column (positive selection), and isolates and flow-throughs were used in Cr assay in parallel with original population. Isolation procedure increased amount of NK cells in total population from initial 4-7% to 25, 15, 45 and 25% in isolates from control, 24 hrs, 72-single and 72-double groups respectively. Although NK isolate produced slightly higher levels of Cr release than initial fraction even at 10 times lower E:T ratio, it might not be entirely valid reason for identifying NK cells as major effector population. But, the fact that at the same time flow through is practically inactive at the same E:T ratio as initial fraction, supports this conclusion.

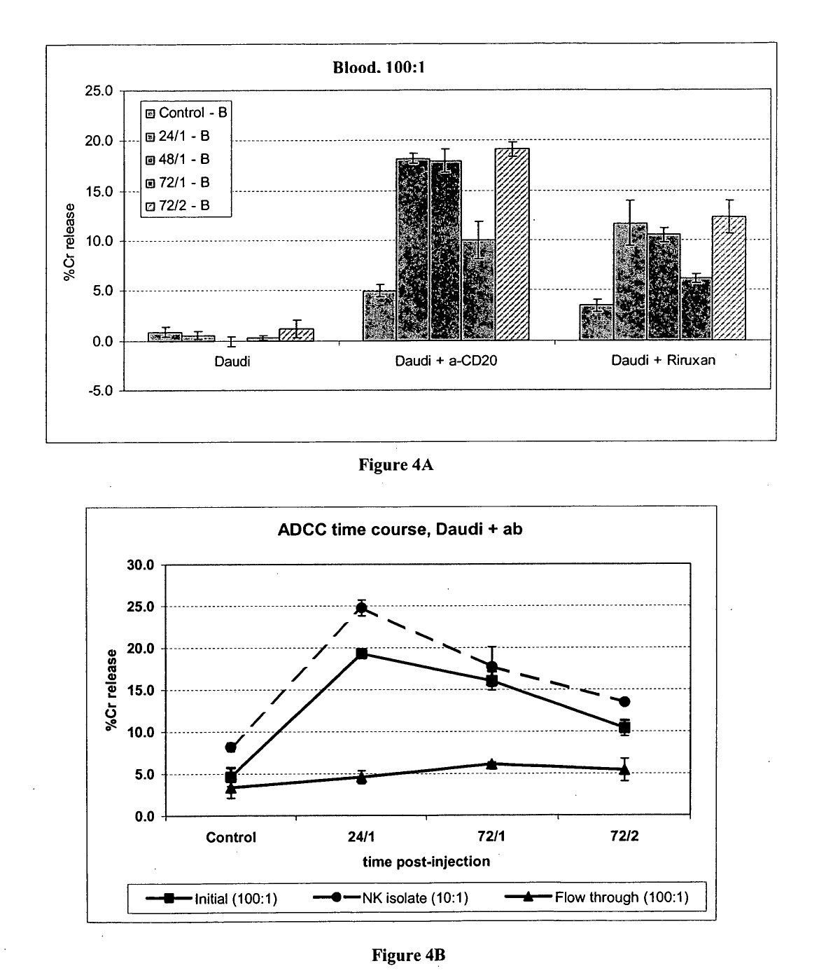

IV administration of cationic liposomes comprising ODN 1m [SEQ ID NO:4] increases NK cells' ability for ADCC, as demonstrated against Daudi cells (murine and humanized Ab), and to less extent against SKBR-3 cells (humanized Ab) (FIG. 4A). Comparative performance of Rituxan™ and murine anti-CD20 Ab is different for effectors from different organs: murine Ab is superior to Rituxan™ in its ability to mediate ADCC for blood cells, slightly superior for spleen and liver and there is no difference for LN. Levels of ADCC induced are the highest in liver, followed by spleen and blood, with the lowest levels in LN. ADCC development in three days time course is similar for effectors from all four organs: elevation at 24 and 48 hours followed by decline (although still higher than control) at 72 hours. Second injection of formulation restored ADCC levels of liver and blood cells to the levels of 24-48 hours time points, but did not improve ADCC for LN, and lead to further decline for spleen cells. Overall increase in ADCC upon formulation injection was up to 10-15% for Rituxan™ and up to 15-20% for murine anti-CD20, depending on source of effector cells.

Major effector population for ADCC in Daudi/anti-CD20 systems appears to be NK cells (FIG. 4B). In order to identify the effector population with highest impact on ADCC, splenocytes from control and several time points were run through NK cells isolation column (positive selection), and isolates and flow-throughs were used in Cr assay in parallel with original population. Isolation procedure increased amount of NK cells in total population from initial 4-7% to 25, 15, 45 and 25% in isolates from control, 24 hrs, 72-single and 72-double groups respectively. Daudi: Although NK isolate produced slightly higher levels of Cr release than initial fraction even at 10 times lower E:T ratio, it might not be entirely valid reason for identifying NK cells as major effector population. But, the fact that at the same time flow through is practically devoid of activity at the same E:T ratio as initial fraction, supports this conclusion.

### EXAMPLE 3

This series of experiments was designed to evaluate NK and LAK activity and ability to mediate ADCC in tumour-free and tumour-bearing mice.

Mice. In this experiment, 20 C57BI/6J female mice at 8-9 weeks old (20-22 g) were used. The animals were housed in groups of 5.

Dosages. There were 4 treatment groups. In two of the groups, each mouse received 105 B16/BL6 cells in 200 ml PBS (IV). 10 days later mice from tumour-free and tumour-bearing treatment groups received an intravenous (i.v.) tail vein injection of cationic liposomes comprising ODN 1m [SEQ ID NO:4]; volume based upon body weight (200 ml for a 20 g mouse, 250 ml for a 25 g mouse, etc). Animals received 20 ODN/kg dose of cationic liposomes comprising ODN 1m [SEQ ID NO:4] per injection; formulation was prepared at 2 mg/ml in HBS.

Harvest. Animals were terminated 48 hours later and organs (spleen, blood, and lung) harvested (sterile conditions not required). Sterile conditions were not required. Tissues were dissociated and cells collected for in vitro analysis.

Data Analysis. Blood, splenic cells (original population, NK isolates and flow-throughs) were used in CTL/ADCC assays against YAC-1, Daudi target cells in the presence and absence of Rituxan™; and analysed on FACS for NK and Monocytes/Macrophages activation (DX5/CD11b/Mac-3 and CD16/CD69/IL12-R). Plasma samples were tested in ELISA for IFNg and IL-12.

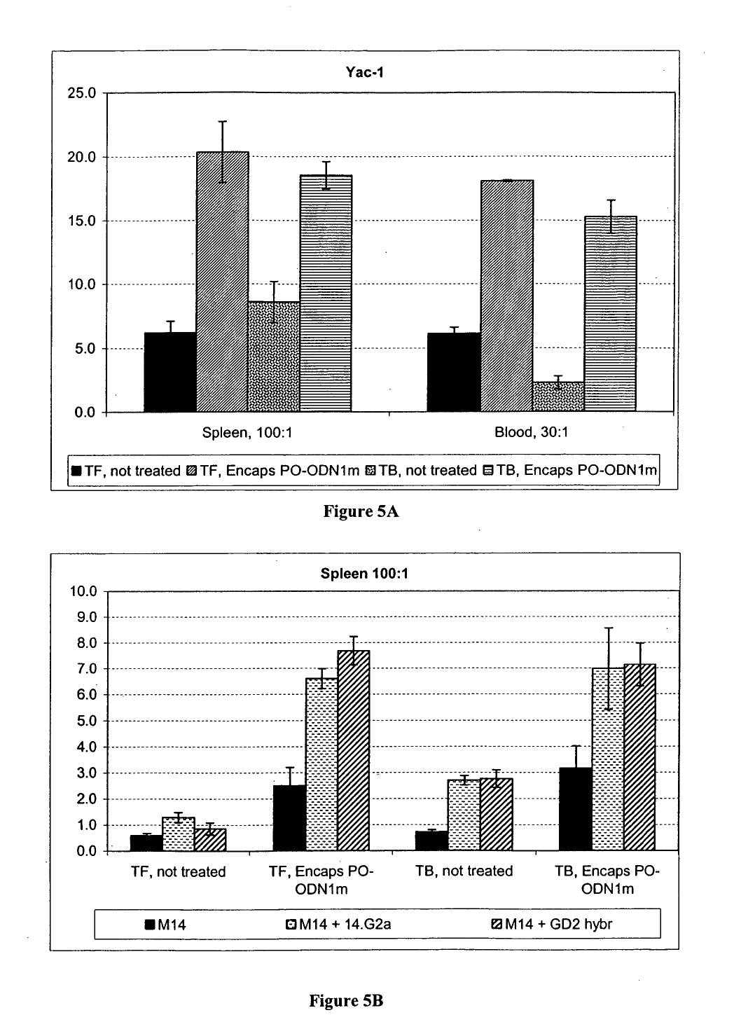

Results. In vitro cytotoxicity. Injection of cationic liposomes comprising ODN 1m [SEQ ID NO:4] induced comparable activation of NK cells, as measured by in vitro cytotoxicity levels against Yac-1 target, in both tumour-free and tumour-bearing animals. The basal levels of Yac-1 killing were similar for splenic NK cells of TF and TB groups; as for blood NK cells, their activity against Yac-1 was slightly lower in TB group (FIG. 5A). Injection of cationic liposomes comprising ODN 1m [SEQ ID NO:4] also stimulated direct and Ab-mediated in vitro killing of M14 cells (human melanoma) (FIGS. 5B and 5C). In spleen, there was increased level of ADCC in TB-control group compared to TF-control. Levels of stimulation of direct and Ab-mediated killing upon treatment with cationic liposomes comprising ODN 1m [SEQ ID NO:4] were similar in TB and TF animals. In blood, there was no difference in TB and TF basal levels of direct and Ab-dependent cytotoxicity against M14 cells; while treatment with cationic liposomes comprising ODN 1m [SEQ ID NO:4] induced higher level of direct and lower level of Ab-dependent killing in TB mice.

### EXAMPLE 4

This series of experiments was designed to investigate the injection dose and regimen (cationic liposomes comprising ODN 1m [SEQ ID NO: 4]) for NK and LAK activity and ability to mediate ADCC (C3H mice).

Mice. In this experiment, 50 C3H female mice from 8-9 weeks old (22-25 g) were used. The animals were housed in groups of 5.

Dosages. One treatment group, with a control, at various time points. Administrations of test samples and controls were via intravenous tail vein injections with injection volume dependent on body weight (e.g., 200 ml for a 20 g mouse, 350 ml for a 25 g mouse, etc.). Animals received 10/20/30/40 mg ODN/kg dose of cationic liposomes comprising ODN 1m [SEQ ID NO:4]; prepared at 1, 2, 3 and 4 mg/ml in HBS.

Harvest. Mice blood and spleen were harvested. Sterile conditions were not required. Tissues were dissociated and cells collected for in vitro analysis.

Data Analysis. Blood and splenic cells were used in CTL/ADCC assays against YAC-1, Daudi target cells in the presence and absence of anti-CD20 Ab; and analysed on FACS for NK and Monocytes/Macrophages expansion/activation (DX5/CD11b/Mac-3 and CD16/CD69/IL12-R). Plasma will be used for ELISA for IFNg and IL-12.

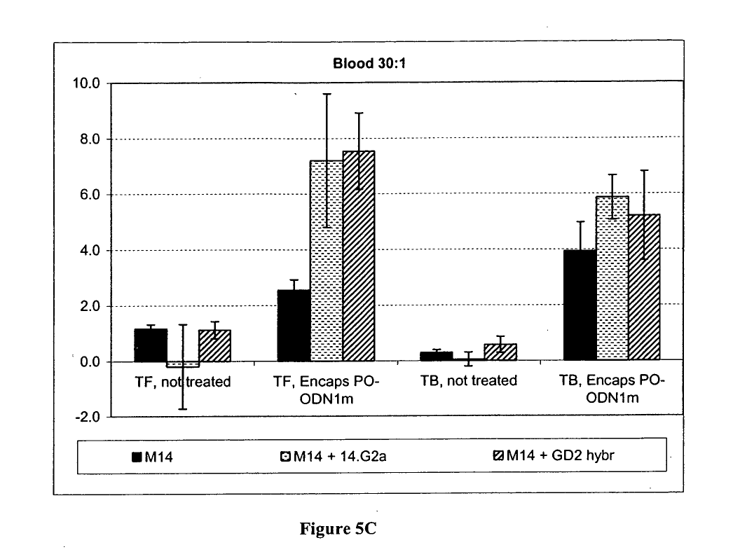

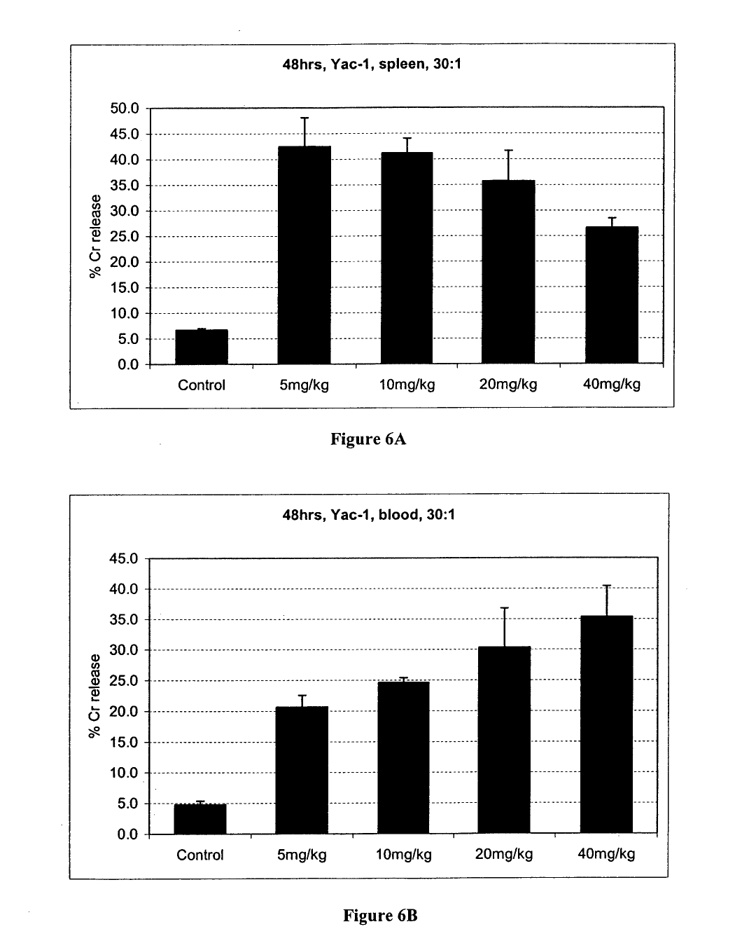

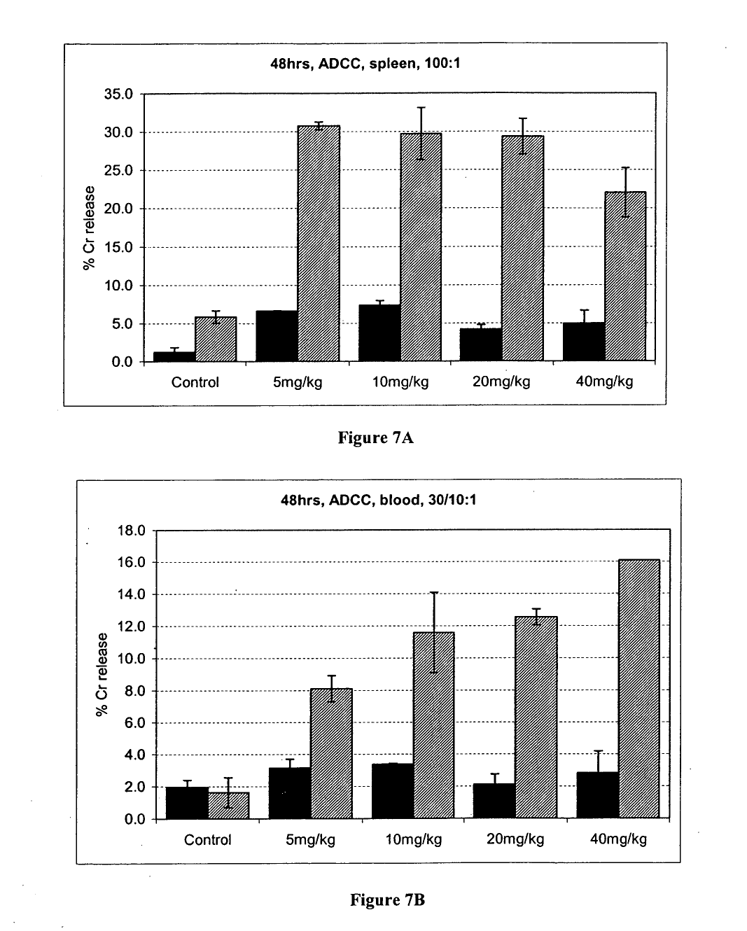

Results. Administration of cationic liposomes comprising ODN 1m [SEQ ID NO: 4] resulted in enhanced NK activity at all doses tested compared to cell activity in untreated animals (FIGS. 6A and B). Increasing IV doses resulted in a parallel increase in blood NK cell activation as measured by in vitro cytolytic activity against Yac-1 target cells (FIG. 6B) as compared to spleen NK cells in which activity was maximal at 5-10 mg/kg and diminished thereafter (FIG. 6A), potentially due to mobilization of cells from the spleen to peripheral blood. The trend in NK activity in both spleen and blood was also reflected in the ability of these cells to mediate ADCC. While increasing doses of cationic liposomes comprising ODN 1m [SEQ ID NO:4] resulted in a parallel increase in ADCC activity against Daudi cells in the presence of an anti-CD20 Ab (FIG. 7B), ADCC activity in spleen NK cells was maximal at 5-20 mg/kg and declined therafter (FIG. 7A). As expected treated animals exhibited dramatically enhanced ADCC activity compared to cells from untreated animals and cytolytic activity from all groups was minimal in the absence of antibody.

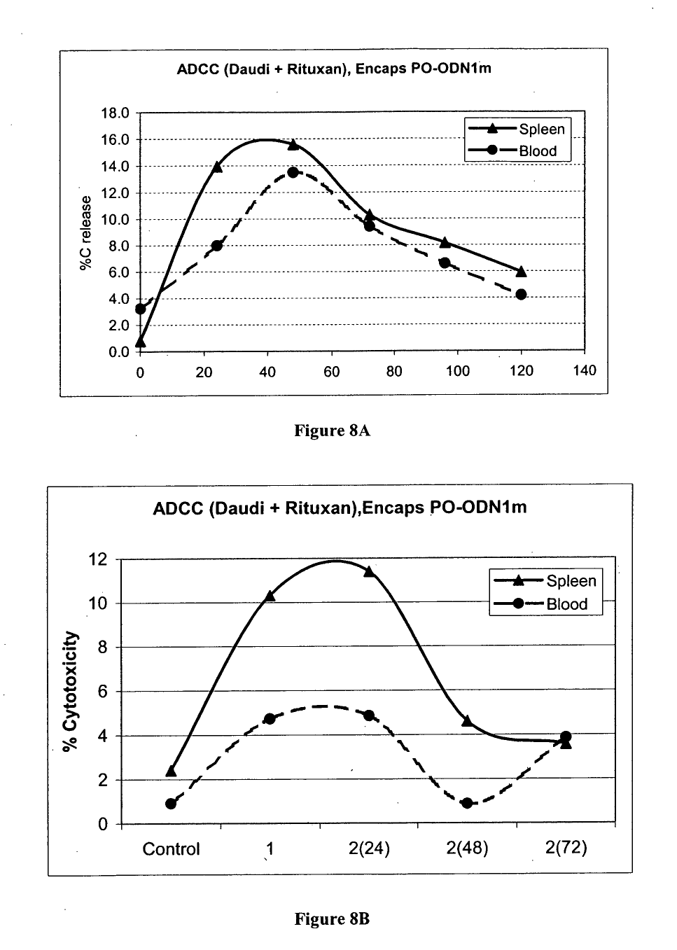

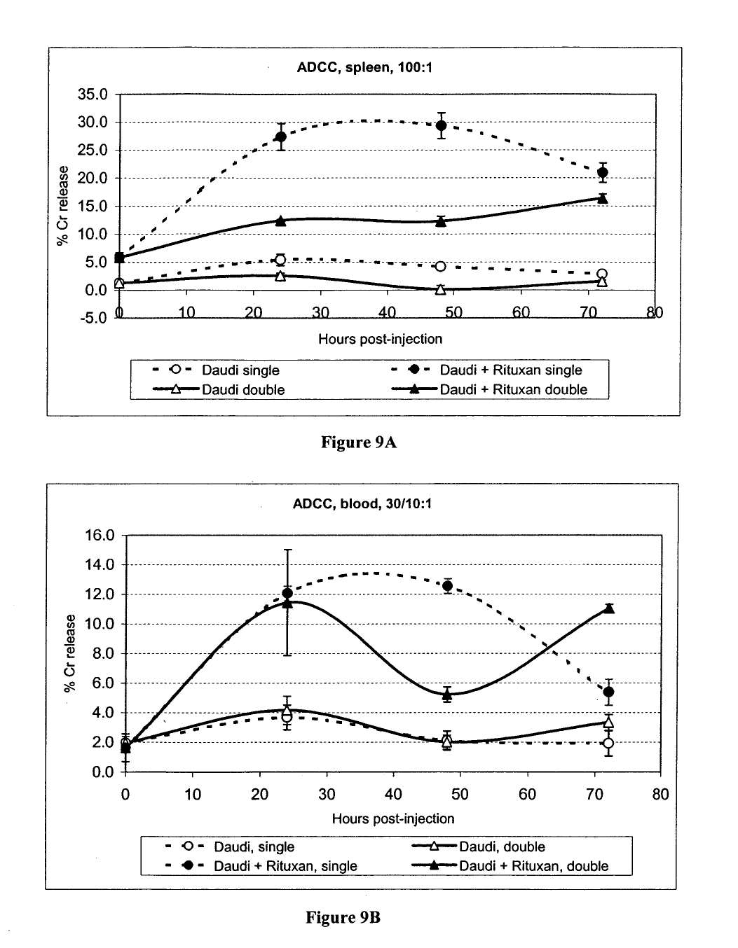

In addition to dose, the effect of cationic liposomes comprising ODN 1m on NK and ADCC activity was also found to be dependent on dosing regimen. Administration of multiple doses of liposomal ODN 1m within a 42-72 hour period did not result in enhanced activity compared to a single dose (FIG. 8). However, multiple doses over a more protracted period did result in some enhanced activity. Administration of cationic liposomal ODN 1m on a weekly dosing regimen was found to result in moderate and significant enhancement of ADCC activity against Daudi cells in the presence of an anti-CD20Ab in spleen (FIG. 9A) and blood (FIG. 9B), respectively. The activity was dependent on the presence of the antibody.

### EXAMPLE 5

This series of experiments was designed to investigate validity of cationic liposomes comprising ODN 1m [SEQ ID NO:4] in combination with Ritiximab in therapeutic model of ADCC.

Mice. In this experiment, 50 SCID C.B-17Balb/c female mice from 6-8 weeks old (20-22 g were used. The animals were housed in groups of 5.

Treatment. There were nine treatment groups, with one control group, at various time points. The control group was challenged IV with 5×10⁶Namalwa cells and treated with HBS. Four treatment control groups received IV challenge of 5×10⁶ Namalwa cells and IV treatment with Rituximab once a week at 5, 10, 20 or 40 mg/dose. One treatment control group was challenged IV with 5×10⁶ Namalwa cells received IV injection of cationic liposomes comprising ODN 1m [SEQ ID NO:4] at 10 mg/kg twice a week. Four treatment groups received IV challenge of 5×10⁶ Namalwa cells and treated with IV injections of cationic liposomes comprising ODN 1m [SEQ ID NO:4] at 10 mg/kg twice a week and Rituximab Ab once a week at 5, 10, 20 or 40 ug/dose.

Dosages. Animals received 10 mg ODN/kg dose of cationic liposomes comprising ODN 1m [SEQ ID NO:4] prepared at 1 mg/mL in PBS.

Tumor Growth. Namalwa cells were cultured for 3-5 passages in vitro prior to the initiation of the experiment. Flasks used in this experiment exhibited 50-60% confluency at harvest. The single cell suspension was transferred to 50 mL conical tubes on ice. Once all cells were harvested, they were washed in 1× sterile Hank's at 1000 rpm, 5 min 40C. Cells were counted and used only if the viability was greater than 90%. Cells were diluted to 5×10⁶ cells per 200 mL (2.5×10⁷cells/mL) in sterile Hank's. The cells were implanted into the mice i.v. (via tail vein) once the cell suspension was warmed up. Care was taken to ensure cells were well mixed prior to inoculation. Mice were checked daily. Body weight measured two times a week.

Data Analysis. Mice were euthanized when they showed signs of morbidity, abdominal distention, hind leg paralysis or weight loss >20%. Mice were terminated by C02 inhalation. Analysis was based on body weight and time to euthanasia. MST (median survival time) was used to determine anti-tumour efficacy as a proof of principal of ADCC in an animal model of cancer. Animals were weighed twice a week. Tolerability and toxicity of the regimen was assessed.

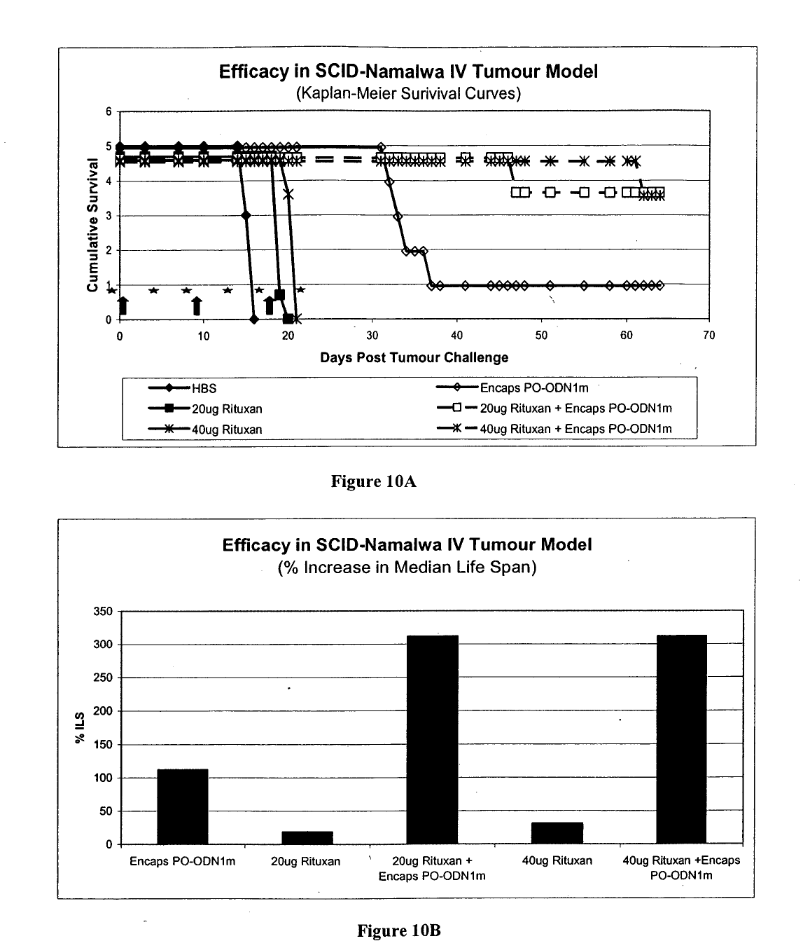

Results. In efficacy studies in SCID mice challenged with the human B-cell lymphoma cell line Namalwa, treatment with a combination of the anti-CD20 Ab Rituxan™ and cationic liposomes comprising ODN 1m resulted in enhanced antitumor efficacy compared to treatment with either Ab or liposomal ODN 1m alone, as judged by enhanced survival (FIG. 10A). Untreated animals had a median survival of 16 days while animals treated with 10 and 20 mg of antibody had median survivals of 20 and 21 days respectively (% increase in life span or % ILS of 25% and 31%) and those treated with liposomal ODN 1m alone had a median survival of 34 days (% ILS of 112%). However, animals treated with a combination of either 10 or 20 mg of Rituxan™ and liposomal ODN 1m had median survivals exceeding 67 days (% ILS >325%) (FIG. 10B).

### EXAMPLE 6

This series of experiments was designed to evaluate, in a syngeneic animal model of cancer (with EL4 tumour cells administered IV in C57BI/6 mice), the anti-tumour efficacy of cationic liposomes comprising an immunostimulatory nucleic acid administered with an anti-GD2 monoclonal antibody for ADCC application.

Mice. In this experiment, 30 C57BI/6 female mice from 10-12 weeks old (20-22 g) used. The animals were housed in groups of 5. There were 6 groups of mice.

Treatment. Animals were challenged IV with 5×10⁴ EL4 cells. One group was Untreated (HBS) and the 5 other received twice a week IV injections of cationic liposomes comprising ODN 1m [SEQ ID NO: 4] at doses of 5 or 10 mg/kg (based on body weight). Three groups received also once a week IV injection of GD2 antibody at 20 mg/mouse (80 ml of 0.250 mg/ml stock).

Dosages. Animals received 5 mg/kg or 10 mg/kg ODN/kg dose of cationic liposomes comprising ODN 1m [SEQ ID NO: 4] per injection; formulation was prepared at 0.5 mg/mL and 1.0 mg/mL in PBS.

Tumor Growth. EL4 cells were cultured for 3-5 passages in vitro prior to the initiation of the experiment. The single cell suspension was transferred to 50 mL conical tubes on ice. Once all cells were harvested, they were washed in sterile Hank's ×1 at 1000 rpm, 5 min 40C. Cells were counted and only used if the viability wass greater than 90%. Cells were diluted to 5×10⁴ cells per 200 mL (2.5×10⁵ cells/mL) in sterile Hank's. The cells were administered IV once the cell suspension was warmed up. Care was taken to ensure cells were well mixed prior to inoculation. Mice were checked daily.

Data Analysis. Mice were euthanized when they showed signs of morbidity, abdominal distention, hind leg paralysis or weight loss >20%. Mice were terminated by CO2 inhalation. Analysis based on survival curve. MST (median survival time) was used to evaluate efficacy of cationic liposomes comprising ODN 1m [SEQ ID NO:4] administered with a monoclonal antibody to exert anti-tumour effects in a syngeneic model of cancer. Animals were weighed twice a week. Tolerability and toxicity of the regimen of cationic liposomes comprising ODN 1m [SEQ ID NO: 4] administration were assessed.

Results. In efficacy studies in the syngeneic C57BI/6-EL4 thymoma IV and SC tumour models, treatment with a combination of an Ab recognizing the tumour associated antigen GD2 and cationic liposomes comprising ODN 1m resulted in enhanced antitumour efficacy compared to treatment with either Ab or liposomal ODN 1m alone. In the C57BI/6-EL4 SC model, treatment with the combination of 20 mg/kg of anti-GD2 antibody and 5 mg/kg liposomal ODN 1m resulted in superior inhibition of tumour growth compared to treatment with equivalent doses of the anti-GD2 antibody or liposomal ODN 1m alone (FIG. 11A). All treatments resulted in inhibition of tumour growth compared to untreated animals. Similarly, in the C57BI/6-EL4 IV tumour model, treatment with the combination of anti-GD2 antibody and liposomal ODN 1m resulted in enhanced efficacy compared to either treatment alone as judged by enhanced survival (FIG. 11B). Untreated animals had a median survival of 17 days while animals treated with anti-GD2 antibody and liposomal ODN 1m alone had median survivals of 24 and 23 days respectively (% ILS of 35 and 41%). Treatment with a combination of Ab and liposomal ODN 1m resulted in a median survival exceeding 31 days (% ILS of greater than 82%) (FIG. 11C).

### EXAMPLE 7

This series of experiments was designed to evaluate an injection regimen (cationic liposomes comprising ODN 1m [SEQ ID NO:4] and ODN 2 [SEQ ID NO: 1] PS free) for NK and LAK activity and ability to mediate ADCC (C3H mice).

Mice. In this experiment, 60 C3H female mice from 8-9 weeks old (22-25 g), by the time experiment, were used. Animals were housed in groups of 5.

Treatment. There was one control and one treatment group, at various time points. Mice received an intravenous (i.v.) tail vein injection with volume based upon body weight (200 ml for a 20 g mouse, 250 ml for a 25 g mouse, etc.).

Harvest. Blood and spleens were harvested. Sterile conditions were not required. Tissues were dissociated and cells collected for in vitro analysis.

Dosages. Animals received 20 mg ODN/kg dose of cationic liposomes comprising ODN 1m [SEQ ID NO:4] per injection; formulation was prepared at 2 mg/ml in PBS.

Data Analysis. Blood and splenic cells were used in CTL/ADCC assays against P815, YAC-1, Daudi target cells in the presence and absence of anti-CD20 ab (Rituxan™ and/or mouse-anti-human IgG1) and SKBR-3 target cells in the presence and absence of Herceptin™; and analysed on FACS for NK and Monocytes/Macrophages activation (DX5/CD16-CD69, CD11b/CD16-CD69, Mac-3/CD16-CD69).

Results. Results shown in FIG. 8B support conclusions drawn in Example 4 regarding the importance of dosing regimen. As seen in FIG. 8A, a single injection of cationic liposomes comprising ODN 1m [SEQ ID NO:4] resulted in enhanced ADCC activity over 5 days, with the peak appearing at 24-48 h post dosing. Administration of two doses within 24, 48 or 72 hours does not alter the kinetics of this stimulation in either blood or spleen, with the enhancement of ADCC activity appearing similar for either a single of double injection.

### EXAMPLE 8

This series of experiments was designed to investigate the ability of cationic liposomes comprising an immunostimulatory nucleic acid to mediate ADCC and facilitate proliferation and mobilization of NK cells using a BrDu incorporation assay.

Mice. 36 C3H female mice, 8-9 weeks old (20-22 g) by the time of the experiment. The animals were housed in groups of 3.

Dosages. There were two treatment groups plus a control, at 2 time points. Administrations of test samples and controls were via intravenous tail vein injections with injection volume dependent on body weight (e.g., 200 ml for a 20 g mouse, 250 ml for a 25 g mouse, etc.). In one group animals received a 20 mg ODN/kg of free ODN 2 [SEQ ID NO:1]. In a second group animals received 20 mg ODN/kg dose of cationic liposomes comprising ODN 1m [SEQ ID NO:4] prepared at 2 mg/ml in PBS. In the control group, animals received HBS. In each of the treated groups there were four sub-groups. Two of the four sub-groups for each treatment regimen were collected at 48 hours, the remaining sub-groups were collected at 168 hours. At each time point, one of the sub-groups was labeled with BrDu for the entire time period and the other sub-group was labeled with BrDu for the final 18 hours of treatment.

Harvest. Blood, bone marrow and spleen were harvested. Tissues were dissociated and cells collected for in vitro analysis.

Formulations. Cationic liposomes comprising ODN 1m [SEQ ID NO:4] were made using the pre-formed vesicle (PFV) technique, and utilized EtOH. The reformulated PFV was extruded through a 200 nm filter atop a 100 nm filter for two passes.

Data Analysis. Cells from all groups was analysed by flow cytometry (FACS) for BrDu incorporation into NK cells. Cells labeled with BrDu for the entire time period, 48 hours or 168 hours, were used to determine the total proliferation of NK cells during the labeling period. As the NK cells divide, the BrDu, a nucleotide analog is incorporated into the newly formed DNA. Cells labeled with BrDu for the final 18 hours of treatment were used to determine the proportion of cells proliferating 48 or 168 hours after treatment.

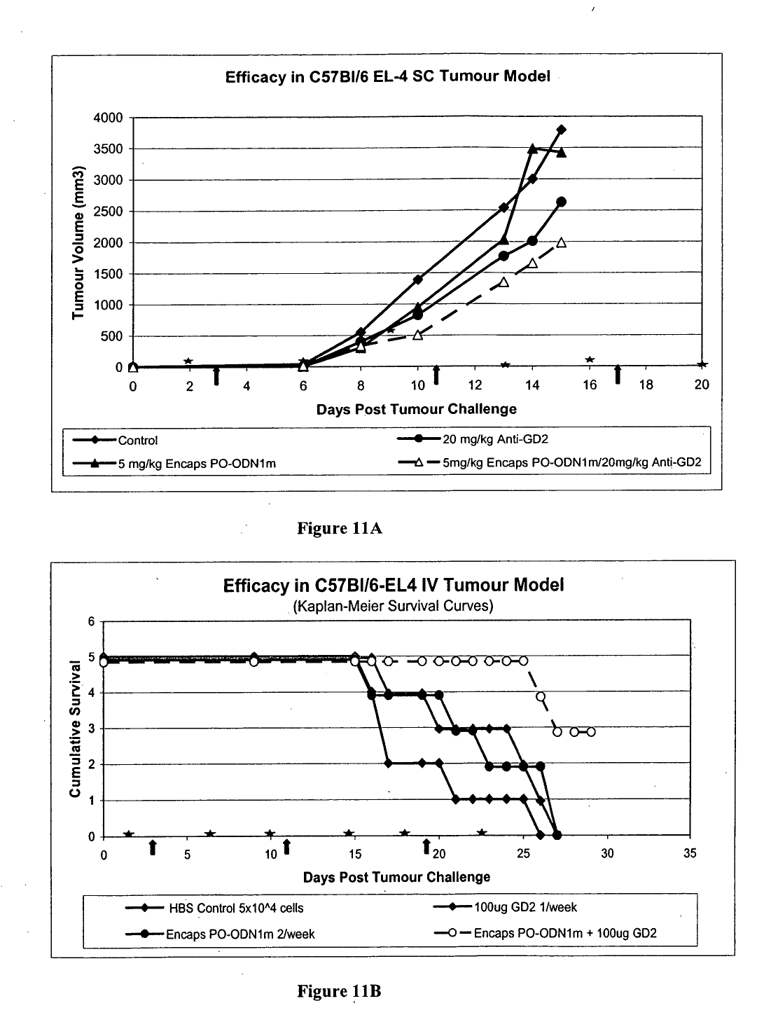

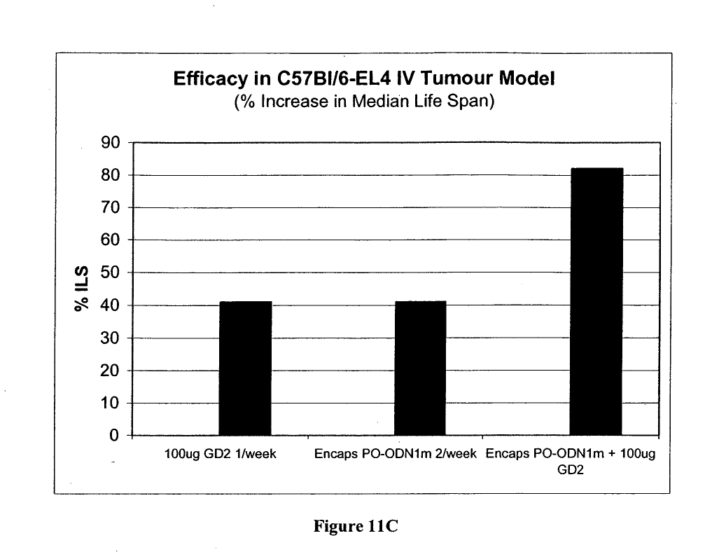

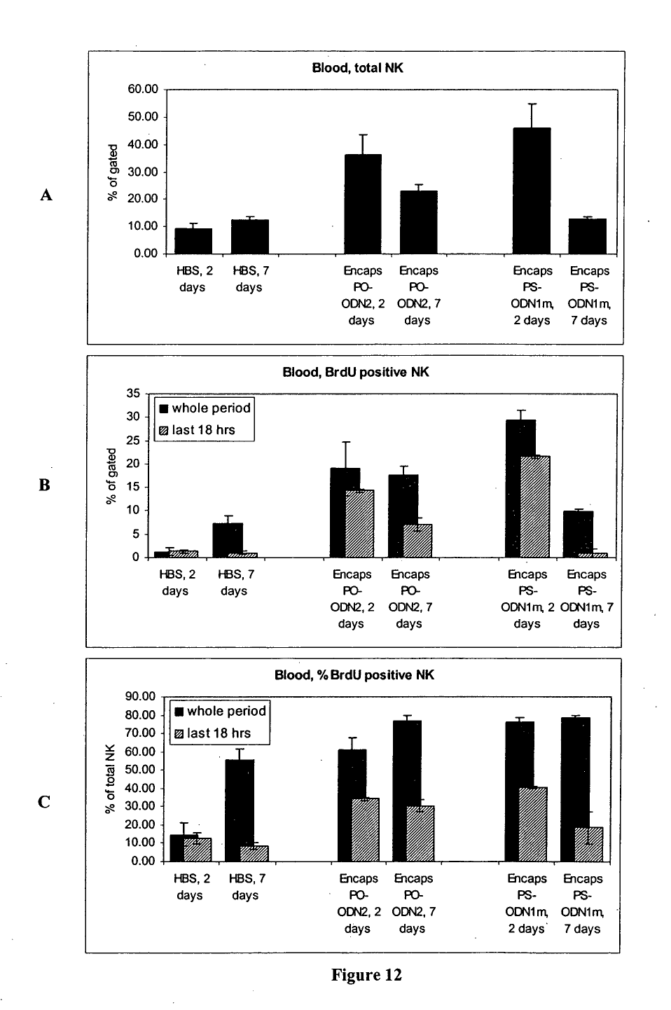

Results. IV administration of cationic liposomes comprising ODN 1m [SEQ ID NO:4] induces expansion of the NK cell population, reflected in increased total NK cells in the blood as compared to the control (FIG. 12A). These data indicate a rapid expansion in the NK cell population by the Day 2 time point, in the peripheral blood. By Day 7, the NK cell population is similar to the control indicating that the majority of the expansion occurs at the earlier 2-day time point and declines to control levels by Day 7.

IV administration of cationic liposomes comprising ODN 1m and free ODN 2 induces NK cell proliferation, reflected in increased BrDu incorporation into NK cells (FIG. 12B). At the Day 2 time point (48 hours) animals treated with liposomal ODN 1m exhibited approximately a 2250% increase in NK cell proliferation over the control (from 1.25% to 29.32%) and over 50% (from 19.01% to 29.32%) increase over the free ODN when the BrDu was present for the entire time period. In addition, during the final 18 hours of treatment, the liposomal ODN 1m exhibited 1472% greater cell proliferation than the control (from 1.37% to 21.54%). Proliferation declined thereafter and by Day 7, NK cell proliferation was only slightly better than the control. Finally FIG. 12C illustrates the percentage of NK cells due to proliferation as compared to the total number of NK cells present in the blood. Approximately 80% of the NK cells present after treatment with cationic liposomal ODN 1m after 2 days are due to proliferation as compared to the control where only 12% are due to proliferation and 60% in the free ODN 2 treated animals.

### EXAMPLE 9

This series of experiments was designed to investigate validity of cationic liposomes comprising ODN 1m [SEQ ID NO:4] in combination with Herceptin™ to enhance ADCC in a therapeutic model of cancer.

Mice. In this experiment, 75 C3H female mice from 8-9 weeks old (20-22 g) were used. The animals were housed in groups of 5.

Treatment. There were 4 treatment groups, with one control group, at various time points. Each group was challenged IV with 103 38C13-Her2/neu cells in 200 ul volume. The control group was treated with HBS. One treatment group was treated with Herceptin™ once a week for three weeks at 50 mg/dose. Another treatment group was treated with an IV injection of cationic liposomes comprising ODN 1m [SEQ ID NO:4] at 20 mg/kg and Herceptin™ Ab at 50 ug/dose once a week. Additional treatment groups received IV injections of one of ODN 1m [SEQ ID NO: 4] free or ODN 2 [SEQ ID NO: 1] PS free at 20 mg/kg.

Tumor Growth. 38C13-Her2/neu cells were cultured for 3-5 passages in vitro prior to the initiation of the experiment. The cells were harvested and single cell suspensions were transferred to 50 mL conical tubes on ice and washed 1× in sterile Hank's at 1400 rpm, 5 min 40C. Cells were counted and were only used if the viability was greater than 90%. Cells were diluted to 1×103 per 200 ul (IV) in sterile Hank's. The cells were implanted into the mice IV once the cell suspension was warmed up. Care will be taken to ensure cells were well mixed prior to inoculation. Mice were checked daily. Body weight was measured two times a week.

Data Analysis. Mice were euthanized when they showed signs of morbidity, abdominal distention, hind leg paralysis or weight loss >20%. Mice were terminated by CO2 inhalation. Analysis was based on body weight and time to euthanasia. MST (median survival time) was used to determine anti-tumor efficacy as a proof of principal of ADCC in an animal model of cancer. Animals were weighed twice a week. Tolerability and toxicity of the regimen was assessed.

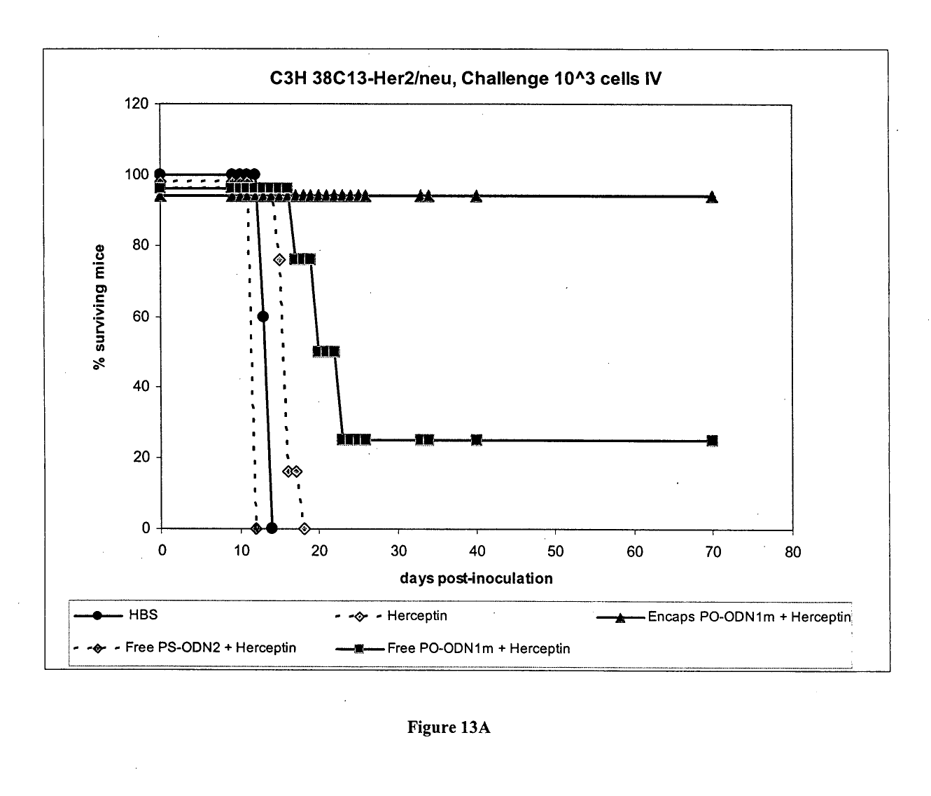

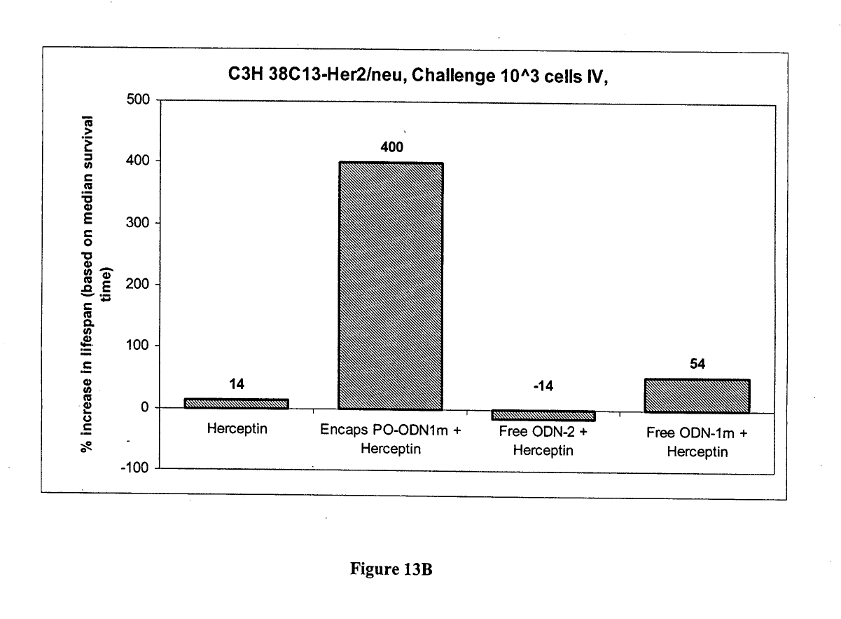

Results. These data show (FIG. 13A) that IV administration of cationic liposomes comprising ODN 1m is effective in enhancing the anti-tumor efficacy of Herceptin™ in this syngeneic tumor model in C3H mice challenged with the murine lymphoma cell line 38C13 that has been transfected to express the human antigen Her2/neu. Administration of Herceptin™ alone at a dose of 50 mg/mouse resulted in a small increase in life span (FIG. 13B) of 14% while administration of 20 mg/kg of free ODN 1m in combination with Herceptin™ at 50 mg/mouse resulted in a small further increase in life span to 54% above control. Surprisingly, administration of 20 mg/kg of free ODN 2 PS in combination with Herceptin™ at 50 mg/mouse did not act to increase lifespan under the conditions tested here. However, administration of 20 mg/kg cationic liposomes comprising ODN 1m in combination with Herceptin™ acted synergistically to enhance anti-tumor efficacy, resulting in an increase in life span of 400% over untreated control. These data show that liposomal ODN 1m acts synergistically with Herceptin™, its activity being superior to either free ODN 2 PS and ODN 1m.

This model is particularly interesting in view of the fact that Her2/neu would be expected to have no functional role in the transfected 38C13 cell line. In human breast and ovarian cancers that are candidates for treatment by Herceptin™, Her2/neu is overexpressed and functions as a receptor that, upon binding of growth factors, transduces proliferative and survival signals resulting in proliferation of the tumor cells. Against these cells, Herceptin™ exerts its anti-tumor effect in two ways, by: 1) blocking growth factor binding and downregulating cell surface expression thus preventing these survival/proliferation signals; and 2) targeting the cells for immune-mediated destruction such as by ADCC. However in the case of these transfected 38C13 cells where the Her2/neu is not expected to have any function as a growth-factor receptor, the anti-tumor effects are most likely directly attributable to ADCC activity alone. Therefore, this model provides strong evidence that ADCC as a single mechanism can exert significant anti-tumor activity and raises the possibility of using monoclonal antibodies that recognize and bind tumor cells but that have no or little therapeutic activity on their own.

### EXAMPLE 10

This series of experiments was designed to investigate validity of cationic liposomes comprising ODN 1m [SEQ ID NO:4] in combination with an anti-GD2 monoclonal antibody to enhance ADCC in a therapeutic model of cancer.

Mice. In this experiment, 30 C57BI/6 female mice from 10-12 weeks old (20-22 g) used. The animals were housed in groups of 5.

Treatment. There were 5 treatment groups, with one control group. Each group was challenged SC with 5×10⁵ EL4 cells. The control group was treated with HBS. Two treatment groups received IV injection of cationic liposomes comprising ODN 1m [SEQ ID NO:4] alone at doses of 5 or 10 mg/kg (based on body weight) twice per week. Three treatment groups received IV injection of cationic liposomes comprising ODN 1m [SEQ ID NO:4] at doses of 5 or 10 mg/kg (based on body weight) twice per week and IV injection of anti-GD2 Ab at 20 ug/dose once a week.

Tumor Growth. Cells were be cultured for 3-5 passages in vitro prior to the initiation of the experiment. The cells were harvested and the single cell suspension were transferred to 50 mL conical tubes on ice and washed 1× in sterile Hank's at 1400 rpm, 5 min 4° C. Cells were counted and were used if the viability is greater than 90%. Cells were diluted to 5×10⁵cells per 200 ul (IV) in sterile Hank's. The cells were implanted into the mice SC once the cell suspension had been warmed up. Care was taken to ensure cells were well mixed prior to inoculation. Mice were checked daily. Body weight was measured two times a week.

Data Analysis. Primary tumor volume was measured using calipers every other day for the duration of the study. Length (mm), width (mm), and height (mm) measurements were made every other day for the duration of the study. Tumor volumes were calculated from the 2 formula:
Tumor Volume (mm³)=(L×W²)/2
Tumor Volume (mm³)=(L×W×H)×π/6

Mice were terminated when tumor volumes reached approximately 2000 mm³ or about 15 days after tumor cell injection. Animals were observed for any adverse reactions during dosing. Mice were also be euthanized at signs of morbidity, abdominal distention, hind leg paralysis or weight loss >20%.

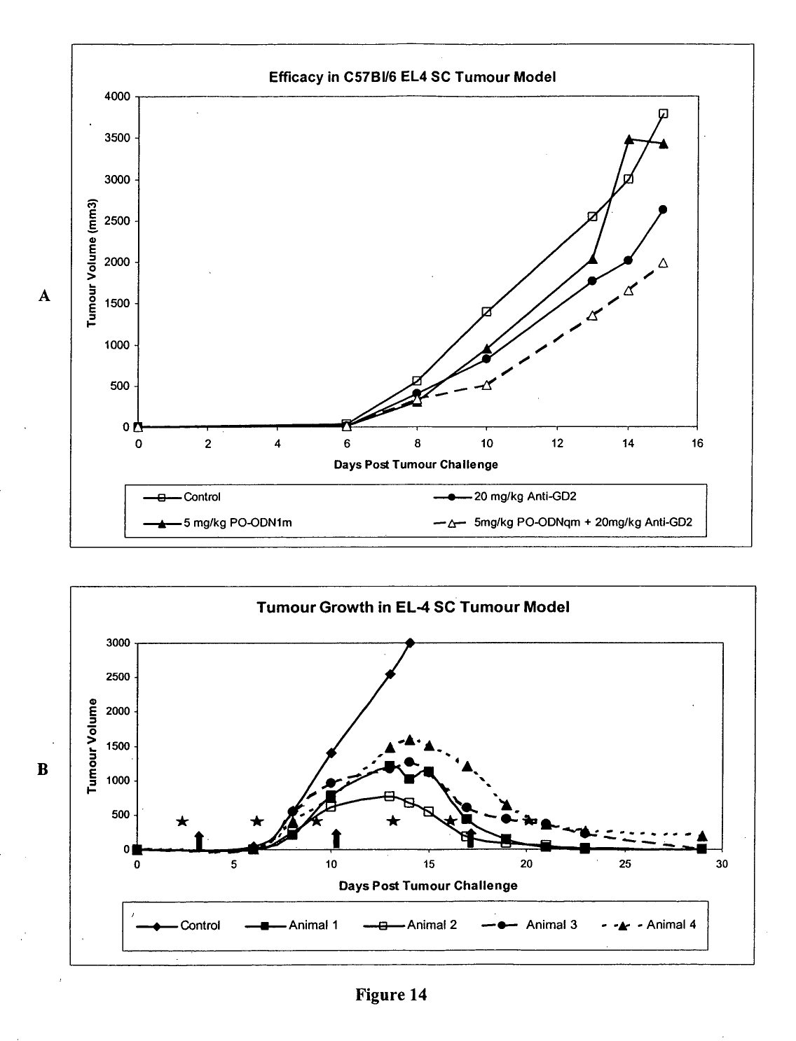

Results. Administration of either anti-GD2 antibody at 20 mg/animal or cationic liposomes comprising ODN 1m at either 10 or 20 mg/kg alone results in only a moderate inhibition in tumor growth. Administration of liposomal ODN 1m at either 10 or 20 mg/kg in combination with anti-GD3 Ab at 20 mg/animal resulted in a moderate enhancement of anti-GD2 anti-tumor activity compared to control animals and those treated with anti-GD2 or liposomal ODN 1m alone (FIG. 14A). Interestingly, although only a moderate enhancement of activity was seen, a relatively high frequency of tumor regression was observed in 25-60% of animals receiving liposomal ODN 1m, both in the presence and absence of Abs (FIG. 14B). The kinetics of tumor growth followed by tumor regression suggested the development of adaptive immune responses that may have been ultimately responsible for the complete regression of the tumor. To assess whether this was the case, animals with regressed tumors were analyzed for Ag-specific cellular and humoral immune responses.

### EXAMPLE 11

This series of experiments was designed to investigate the ability of cationic liposomes comprising ODN 1m [SEQ ID NO: 4] in combination with anti-GD2 monoclonal antibody to enhance ADCC in a therapeutic model of cancer and to facilitate the development of secondary immune responses.

Mice. Animals surviving after treatment described in Example 10.

Harvest. Mice spleen and plasma were harvested. Tissues were disassociated and cells collected for in vitro analysis.

Data Analysis Harvested splenocytes were stimulated in vitro with mitomycin C treated EL4 tumor cells. Splenocytes were then analyzed by Cr release assay for in vitro ability to kill tumor cells. Plasma samples were tested by flow cytometry (FACS) for the presence of Ab that are able to recognize and bind to tumor cells.

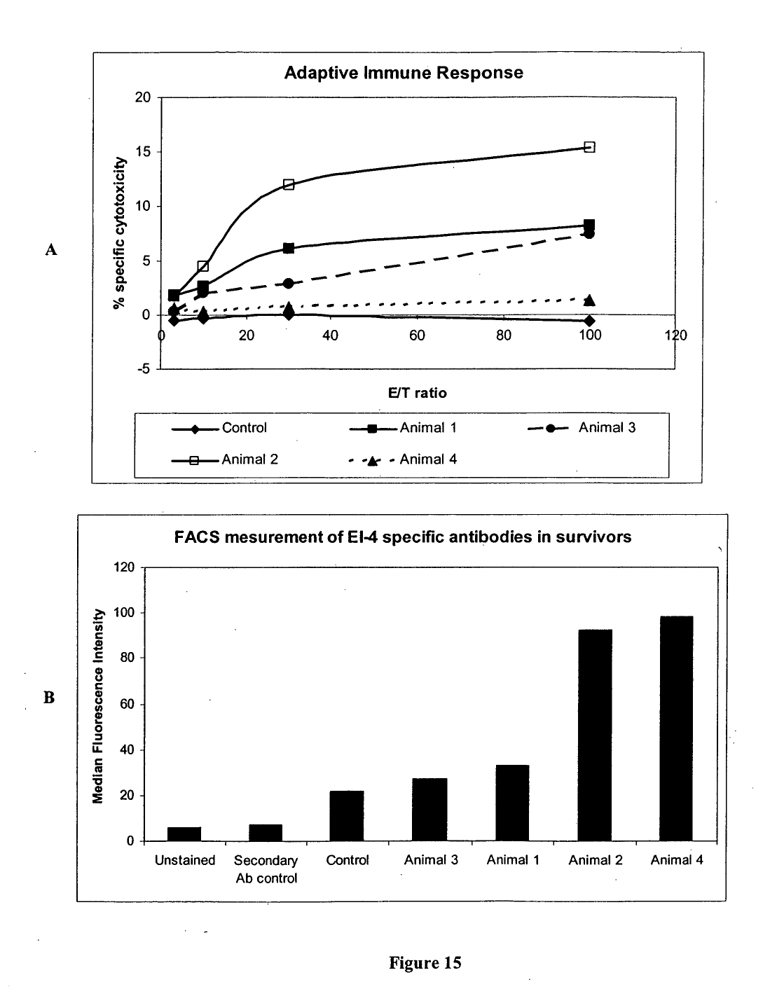

Results Data from these studies indicate the development of secondary adaptive immune responses both in terms of antigen-specific cellular and humoral responses. Splenocytes which had been isolated from animals in which SC administered EL-4 tumors had completely regressed following treatment with a combination of liposomal ODN 1m, and stimulated in vitro, demonstrated enhanced ability to lyse EL-4 tumor cells in an Ag-specific manner in a chromium release assay compared to splenocytes from naive animals as shown in FIG. 15A. Furthermore, serum isolated from these same animals and analyzed by flow cytometry revealed the presence of immunoglobulins that were able to recognize and bind EL-4 tumor cells, FIG. 15B. Both of these results indicate the development of secondary antigen-specific, anti-tumor adaptive immune responses in those animals able to completely clear the initial tumor challenge. These data indicate that treatment with liposomal ODN 1m in combination with a tumor-specific Ab can result in the development of long-lasting, antigen-specific adaptive immune responses and raise the possibility of developing long-term protection from disease relapse.

### EXAMPLE 12

This series of experiments was designed to evaluate, in a syngeneic animal model, the antitumor efficacy of cationic liposomes comprising ODN 1m [SEQ ID NO: 4] administered with an anti-PS monoclonal antibody to enhance ADCC in a therapeutic model of cancer.

Mice. In this experiment, 38 C57BI/6 female mice from 10-12 weeks old (20-22 g) used. The animals were housed in groups of 5.

Treatment. There were 5 treatment groups, with one control group. Each group was challenged SC with 1×10⁵ EL4 cells. The control group was treated with HBS. One treatment group received an IV injection of 10 mg/kg cationic liposomes comprising ODN 1m [SEQ ID NO: 4] (based on body weight) twice per week. One treatment group received IV injections of anti-PS Ab at 50 ug/ml once per week. Additional treatment groups received IV injection of 10 mg/kg cationic liposomes comprising ODN 1m [SEQ ID NO: 4] (based on body weight) twice per week and IV injection of either anti-PS2 Ab or Herceptin™ at 50 ug/dose once a week.

Tumor Growth. EL4 cells were cultured for 3-5 passages in vitro prior to the initiation of the experiment. The single cell suspension were transferred to 50 mL conical tubes on ice. Once all cells were harvested, they were washed in sterile Hank's ×1 at 1000 rpm, 5 min 4° C. Cells were used if the viability is greater than 90%. Cells were diluted to 105 cells per 100 mL (1×106 cells/mL) in sterile Hank's. The cells were administered sc once the cell suspension was warmed up. Care was taken to ensure cells were well mixed prior to inoculation. Mice were checked daily.

Data Analysis. Animals were observed for any adverse reactions during dosing. Primary tumor volume was measured using calipers. Length (mm), width (mm), and height (mm) measurements will be made every other day for the duration of the study. Tumor volumes were calculated from the 2 formula:
Tumor Volume (mm³)=(L×W²)/2
Tumor Volume (mm³)=(L×W×H)×π/6

Mice were terminated when tumor volumes reached approximately 2000 mm³ or about 15 days after tumor cell injection or on the judgment of vivarium staff. Mice were also euthanized if they showed signs of morbidity, abdominal distention, hind leg paralysis or weight loss >20%. Mice were terminated by CO2 inhalation.

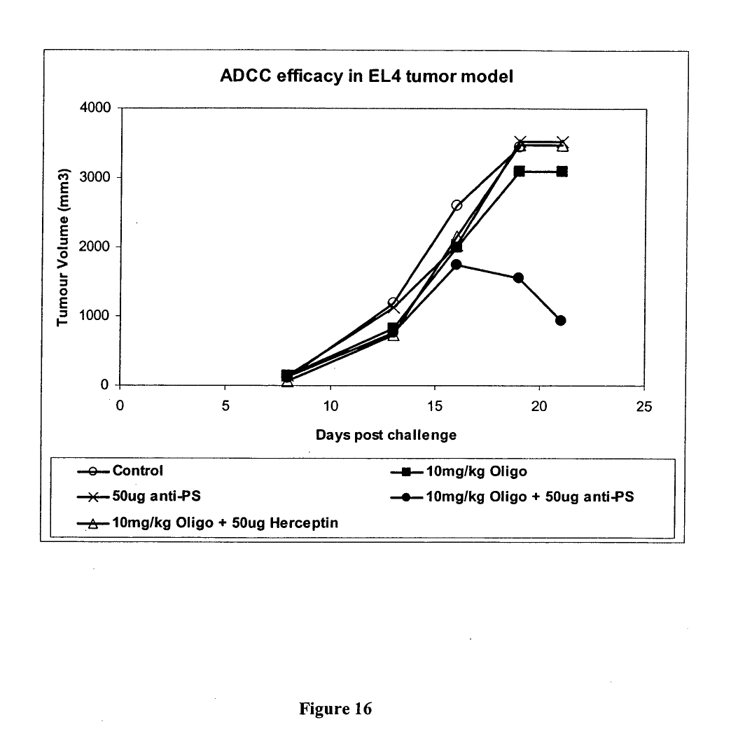

Results. These data show that IV administration of cationic liposomes comprising ODN 1m is effective in enhancing the anti-tumor efficacy of an anti-angiogenic antibody in this syngeneic sc tumor model using the murine thymoma cell line EL-4 in a C57BI/6 mice. The antibody is specific for phosphatidylserine (PS), a lipid that is found to be highly expressed on both tumor vasculature as well as tumor cells. Administration of the anti-PS antibody alone at a dose of 50 mg/mouse did not have any appreciable effect on tumor growth. Similarly, administration of liposomal ODN 1m alone at a dose of 10 mg/kg resulted in only a modest inhibition of tumor growth. However, administration of the anti-PS antibody at 50 mg/mouse in combination with liposomal ODN 1m at 10 mg/kg resulted in significant inhibition of tumor growth as shown in FIG. 16. In fact, after the average tumor volume increased to approximately 1800 mm³ by day 16, regression of the tumor was observed, with average volume declining to 1000 mm³ by day 21 and ultimately resulting in complete elimination of detectable tumor.

### EXAMPLE 13

This series of experiments was designed to investigate validity of cationic liposomes comprising ODN 1m [SEQ ID NO: 4] in combination with anti-PS Ab to enhance ADCC in a therapeutic model of cancer.

Mice. In this experiment, 72 C3H female mice from 8-9 weeks old (20-22 g) were used. The animals were housed in groups of 3 or 4.

Treatment. There were 3 treatment groups, with one control group, at various time points. Each group was challenged IV with 3×103 38C13 cells in 50 ul volume. The control group was treated with HBS. One treatment group was treated with anti-PS Ab at 15 ug/dose once a week for three weeks. Another treatment group was treated with an IV injection of cationic liposomes comprising ODN 1m [SEQ ID NO: 4] at 20 mg/kg and anti-PS Ab at 15 ug/dose once a week. A further treatment group received IV injections of cationic liposomes comprising ODN 1m [SEQ ID NO: 4] at 20 mg/kg.

Tumor Growth. Cells were passage 31 by the time of the initiation of the experiment. The cells were harvested and the single cell suspension was transferred to 10 mL conical tubes on ice and washed 1× in sterile PBS at 1000 rpm, 5 min 4° C. Cells were used if the viability was greater than 90%. Cells were be diluted to 3×10³ cells per 50 ul (20×10³ and 60×10³ cells/ml) in sterile PBS (2 ml for each concentration). The cells were implanted into the mice SC once the cell suspension has been warmed up. Care was taken to ensure cells were well mixed prior to inoculation. Mice were checked daily. Tumour size and body weight were be measured two times a week.

Data Analysis. Primary tumor volume was measured using calipers every other day for the duration of the study. Mice were terminated when tumor volumes reached approximately 2000 mm³ (L×W×W/2) or on the judgment of vivarium staff. Mice were monitored and were euthanized upon signs of disease progression.

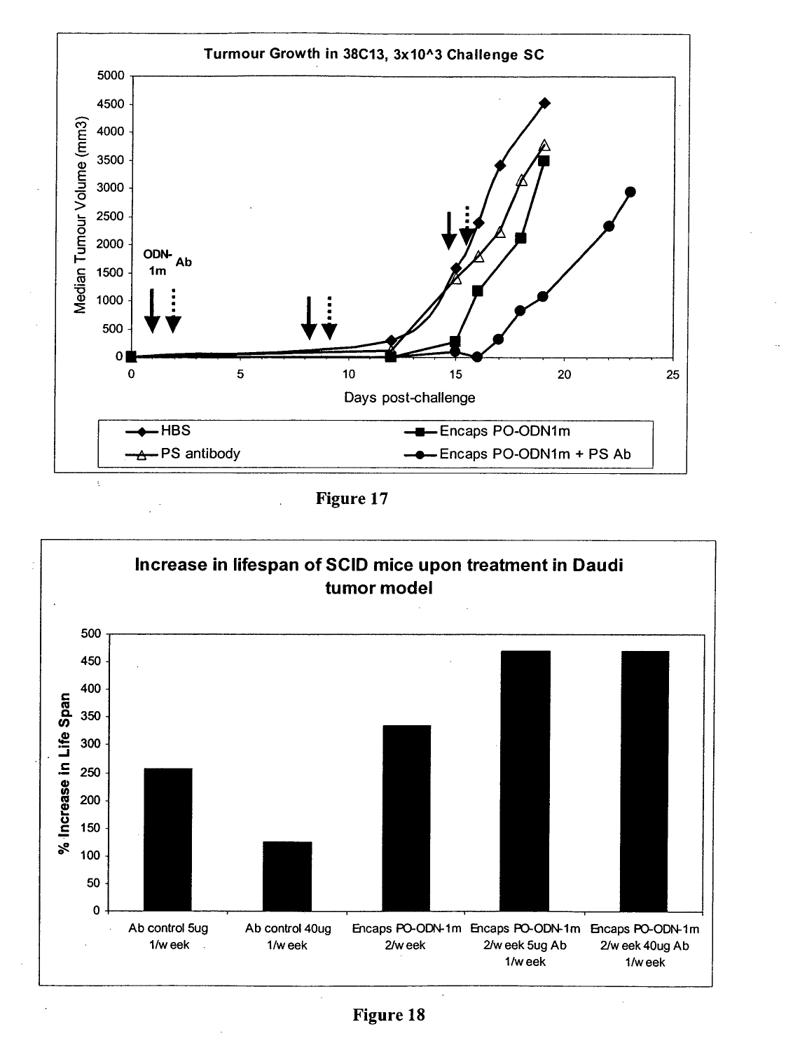

Results. These data show that IV administration of cationic liposomes comprising ODN 1m is effective in enhancing the anti-tumor efficacy of an anti-angiogenic antibody in this syngeneic sc tumor model using the murine lymphoma cell line 38C13 in C57BI/6 mice. The antibody is specific for phosphatidylserine (PS), a lipid that is found to be highly expressed on both tumor vasculature as well as tumor cells, FIG. 17. Administration of the anti-PS antibody alone at a dose of 15 mg/mouse and liposomal ODN 1m alone at a dose of 10 mg/kg were both found to exert modest inhibitory effects on tumor growth compared to untreated control animals. However, administration of the anti-PS antibody at 15 mg/mouse in combination with liposomal ODN 1m at 10 mg/kg resulted in significant inhibition of tumor growth compared to both untreated control animals and animals treated with either agent alone.

### EXAMPLE 14

This series of experiments was designed to investigate validity of cationic liposomes comprising ODN 1m [SEQ ID NO: 4] in combination with Rituximab to enhance ADCC in a therapeutic model of cancer.

Mice. In this experiment, 50 SCID C.B-17 Balb/c female mice from 6-8 weeks old (20-22 g) were used. The animals were housed in groups of 5.

Treatment. There were 3 treatment groups, with two-antibody control group. Each group was challenged IV with 5×10⁶ Daudi cells. The control groups were treated with 5 ug/dose and 40 ug/dose once per week. One treatment group received an IV injection of cationic liposomes comprising ODN 1m at 10 mg/kg twice a week. Additional treatment groups received IV injections of cationic liposomes comprising ODN 1m at 10 mg/kg twice a week and Rituximab Ab at either 5 ug/dose or 40 ug/dose once a week.

Tumor Growth. Daudi cells were cultured for 3-5 passages in vitro prior to the initiation of the experiment. Flasks used in this experiment exhibited 50-60% confluency at harvest. The single cell suspension was transferred to 50 mL conical tubes on ice. Once all cells were harvested, they were washed in 1× sterile Hank's at 1000 rpm, 5 min 40C. Cells were used if the viability was greater than 90%. Cells were diluted to 5×10⁶ cells per 200 mL (2.5×107 cells/mL) in sterile Hank's. The cells were implanted into the mice IV (via tail vein) once the cell suspension had warmed up. Care was taken to ensure cells were well mixed prior to inoculation. Mice were checked daily. Body weight was measured two times a week.

Data Analysis. Mice were euthanized when they showed signs of morbidity, abdominal distention, hind leg paralysis or weight loss >20%. Mice were terminated by CO2 inhalation. Analysis was based on body weight and time to euthanasia. MST (median survival time) was used to determine anti-tumour efficacy as a proof of principal of ADCC in an animal model of cancer. Animals were weighed twice a week. Tolerability and toxicity of the regimen was assessed.

Results. These data show that IV administration of cationic liposomes comprising ODN 1m is effective in enhancing the anti-tumor efficacy of Rituxan™ in this xenogeneic tumor model using the human B-cell lymphoma cell line Daudi in SCID mice. Administration of Rituxan™ alone at doses of 5 and 40 mg/mouse was effective in increasing life span by more than 250 and 120% respectively compared to untreated animals while administration of the liposomal ODN 1m at a dose of 10 mg/kg resulted in an increase in life span of almost 350%. However, administration of Rituxan™ at 5 and 40 mg/mouse in combination with liposomal ODN 1m at 10 mg/kg resulted in an enhanced increase in life span of over 450%, FIG. 18. These data are more impressive in light of the fact that the animals in the combination group were euthanized rather than succumbing to malignant disease. All of the animals in both combination groups were in apparent good health with no signs of disease at time of euthanasia. Thus, we could expect that the combination had a much more pronounced effect on life span and that 470% is a very conservative estimate.

### EXAMPLE 15

This series of experiments was designed to investigate the synergy between liposomal ODN and Herceptin™ to inhibit MCF-7 her2/neu tumour growth through enhanced ADCC activity.

Mice. In this experiment, 50 SCID C.B-17Balb/c female mice from 6-8 weeks old (20-22 g were used. The animals were housed in groups of 5.

Treatment. There were 7 treatment groups, with one control group. Each group was challenged SC with 1×10⁷ MCF-7 cells in 50 ul. The control group was treated with HBS. One set of treatment groups received an IV injection of cationic liposomes comprising ODN 1m [SEQ ID NO: 4] at 10 or 20 mg/kg twice a week for 3 weeks. Another treatment group received an IV injection of 10 mg/kg cationic liposomes comprising ODN 1m [SEQ ID NO: 4] and 50 ug irrelevant Ab Rituximab. Another set of treatment groups received one of 50 ug/dose Herceptin™ or 75 ug/dose Herceptin™. Additional treatment groups received IV injections of cationic liposomes comprising ODN 1m [SEQ ID NO: 4] at 20 mg/kg twice a week and Herceptin™ Ab at either 50 ug/dose or 75 ug/dose once a week. One day period to challenging the animals with the tumor cells, each animal was implanted with a 17-b-estradiol tablet as MCF-7 tumor cells require estrogen to grow.

Tumor Growth. MCF-7 cells were cultured for 3-5 passages in vitro prior to the initiation of the experiment. The single cell suspension was transferred to 50 mL conical tubes on ice. Once all cells were harvested, they were washed in sterile Hank's ×1 at 1000 rpm, 5 min 4° C. Cells were only used if the viability was greater than 90%. Cells were diluted to 10×10⁶ cells per 50 mL (200×10⁶ cells/mL) in sterile Hank's. The cells were administered SC once the cell suspension had been warmed up. Care was taken to ensure cells were well mixed prior to inoculation. Tumor size was measured twice per week.

Data Analysis. Mice were euthanized when they showed signs of morbidity, abdominal distention, hind leg paralysis or weight loss >20%. Mice were terminated by CO2 inhalation. MTS (median tumour size) was used to choose optimal dose of ODN 1m [SEQ ID NO: 4] for ADCC development. Animals were weighed twice a week. Tolerability and toxicity of the regimen of ODN 1m [SEQ ID NO: 4] administration was assessed.

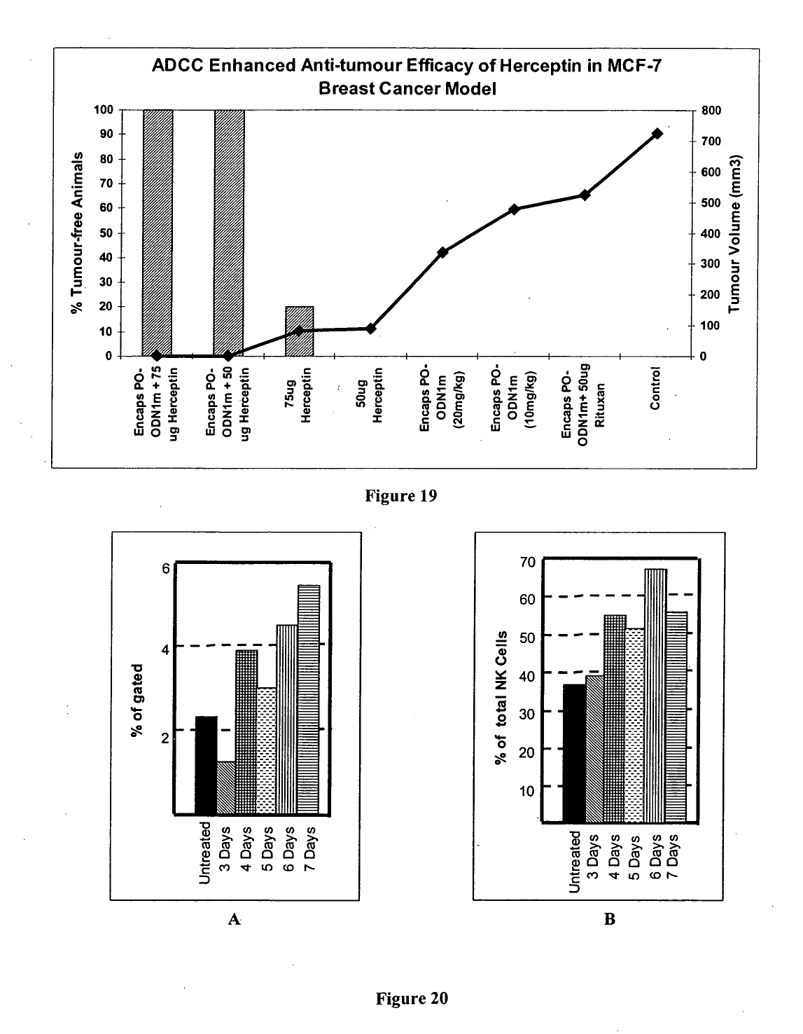

Results. These data show that IV administration of cationic liposomes comprising ODN 1m is effective in enhancing the anti-tumor efficacy of Herceptin™ in this xenogeneic tumor model using the human breast cancer cell line MCF-7 in SCID mice. Administration of Herceptin™ alone at doses of 50 and 75 mg/mouse was effective in reducing tumor size by 87 and 89% respectively compared to untreated control animals while administration of liposomal ODN 1m alone at doses of 10 and 20 mg/kg resulted in a 34 and 54% reduction in tumor size, FIG. 19. However, administration of Herceptin™ at doses of 50 and 75 mg/mouse in combination with liposomal ODN 1m at 20 mg/kg resulted in a complete inhibition of MCF-7 tumor growth, with no detectable tumor. As expected, administration of liposomal ODN 1m at 10 mg/kg in combination with an irrelevant antibody that did not recognize the tumor cells, in this case Rituxan™, did not result in enhanced tumor growth inhibition compared to the cationic liposomal ODN 1m alone at an equivalent dose. In addition, the ability of liposomal ODN 1m to enhance antitumor efficacy of Herceptin™ in this animal model are further demonstrated by the fact that all animals in the control, irrelevant antibody, 10 and 20 mg/kg liposomal ODN 1m and 50 mg/animal Herceptin™ groups as well as 80% of animals in the 75 mg/animal group exhibited tumor burden while all animals in the groups treated with a combination of Herceptin™ and liposomal ODN 1m were completely tumor free.

### EXAMPLE 16

This series of experiments using two independent tumor models was designed to evaluate NK cell migration to the tumour site.

EL-4 Tumor Model

Mice. In this experiment, 65 C57BI/6J female mice from 8-9 weeks old (20-22 g) w used. The animals were housed in groups of 5.

Treatment. There were 2 treatment groups. Each group was challenged SC with 5×105 EL4 cells in 50 ul PBS. The first treatment group was tumor bearing and treated with HBS. The second group was tumor bearing and received an IV injection of 20 mg/kg cationic liposomes comprising ODN 1m [SEQ ID NO: 4].

Harvest. Tumours were harvested, no sterile conditions required. Tissues were dissociated and cells collected for in vitro analysis.

Formulations. Cationic liposomes comprising an immunostimulatory nucleic acid were made using the pre-formed vesicle (PFV) technique, and utilized EtOH. The reformulated PFV was extruded through a 200 nm filter atop a 100 nm filter for two passes.

Data Analysis. Cells from the tumor were analysed by flow cytometry (FACS) for activation of NK cell number (by DX5 expression) and activation status (by CD16 expression).

38C13 Tumor Model

Mice. In this experiment, 30 CH3 female mice from 8-9 weeks old (20-22 g) were used. The animals were housed in groups of 3.

Treatment There were 3 treatment groups. The first group was tumor free and received an IV injection of 20 mg/kg cationic liposomes comprising ODN 1m [SEQ ID NO: 4] once per week. Each tumor bearing group was challenged SC with 1×10⁶ 38C13 cells pretreated with MMC (mitomycin C) in 100 ul PBS. One group of tumor bearing mice was treated with HBS. The second group of tumor bearing mice received an IV injection of 20 mg/kg cationic liposomes comprising ODN 1m [SEQ ID NO: 4] (based on body weight) once per week.

Harvest. Peritoneal washes, no sterile conditions required. Tissues were dissociated and cells collected for in vitro analysis.

Formulations. Cationic liposomes comprising ODN 1m [SEQ ID NO: 4] were made using the pre-formed vesicle (PFV) technique, and utilized EtOH. The reformulated PFV was extruded through a 200 nm filter atop a 100 nm filter for two passes.

Data Analysis. Cells from peritoneal washes were analysed by flow cytometry (FACS) for NK cell number (by DX5 expression) and activation status (by CD69 expression)

Results. Data from these two studies indicate that IV administration of cationic liposomes comprising ODN 1m [SEQ ID NO: 4] results in homing of NK cells to sites of tumor burden thus effectively increasing the number of these immune effector cells in sites of disease compared to untreated animals. This phenomenon of NK cell homing has been demonstrated in two different animal models. In C57BI/6 animals bearing a SC solid EL-4 tumor, enhanced levels of activated NK cells (as assessed by DX-5 NK phenotype marker and CD16 activation marker expression) were detected in tumor tissue 4-7 days after treatment as compared to untreated animals, accounting for as high as 5.3% of cells in the tumor compared to just 2.3% in control animals, FIG. 20A. Similarly, evaluation of the activation status of NK cells in the tumor also demonstrated that iv administration resulted in enhanced activation of NK cells within the tumor, with as high as 66% of NK cells within the tumor (FIG. 20B) being activated after administration of liposomal ODN 1m compared to just 37% in untreated animals.

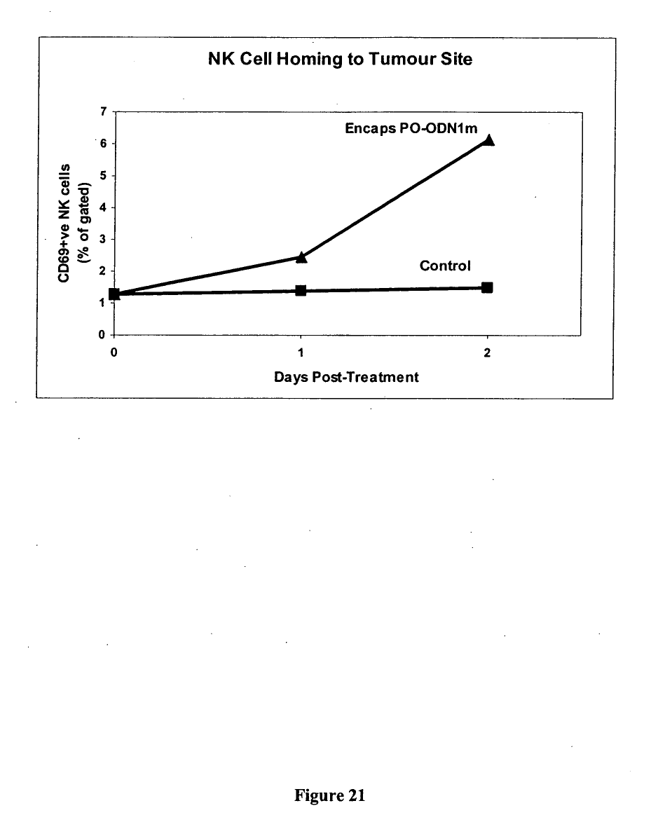

In C3H animals bearing IP 38C13 tumors, evaluation of activated NK cell number (as assessed by DX-5 NK phenotype marker and CD69 activation marker expression) in peritoneal washes also demonstrated enhanced homing to sites of tumor burden following IV administration of cationic liposomal ODN 1m compared to untreated control animals. While the number of activated NK cells remained constant in untreated, tumor-bearing animals at approximately 1.2% of total isolated cells, IV administration resulted in a modest increase in activated NK cell numbers in the peritoneal cavity in tumor-free animals increasing to 3% over 48 h, FIG. 21. However, in tumor-bearing animals, the activated NK cell content increased to approximately 6% over 48 h following liposomal ODN 1m administration.

Data from both of these studies demonstrate that IV administration of cationic liposomes comprising ODN 1m [SEQ ID NO: 4] effectively increases the number of activated NK cells in sites of tumor burden. This observation is relevant, and effectively translates to concentrating the effective immune activity exerted by these cells to sites of disease where they are required and would be most effective.

1. A composition for stimulating an enhanced antibody dependent cellular cytotoxicity response in a subject, comprising a therapeutic antibody in combination with a cationic liposome comprising an immunostimulatory nucleic acid.

2. The composition according to claim 1, wherein said immunostimulatory nucleic acid is an oligodeoxynucleotide (ODN) having at least one CpG dinucleotide.

3. The composition according to claim 2, wherein the cytosine in said CpG dinculeotide is methylated.

4. The composition according to claim 1, wherein said immunostimulatory nucleic acid comprises the nucleic acid sequence 5′ TAAZGTTGAGGGGCAT 3′ (ODN1m) (SEQ ID NO:4).

5. The composition according to claim 1, wherein said immunostimulatory nucleic acid comprises the nucleic acid sequence 5′ TCCATGAZGTTCCTGAZGTT 3′ (ODN2m) SEQ ID NO:32).

6. The composition according to claim 1, wherein said cationic liposome fully encapsulates said nucleic acid.

7. The composition according to claim 1, wherein said therapeutic antibody is an anti-CD20 monoclonal antibody.

8. The composition according to claim 7, wherein said anti-CD20 monoclonal antibody is Rituxan™.

9. The composition according to claim 1, wherein said therapeutic antibody is an anti-Her2/neu antibody.

10. The composition according to claim 9, wherein said anti-Her2/neu antibody is Herceptin™.

11. A mammalian NK cell activated ex vivo or in vivo by a cationic liposome comprising an immunostimulatory nucleic acid, wherein said activated NK cell is bound to the Fc portion of a therapeutic antibody directed to a tumor-associated antigen.

12. The composition according to claim 11, wherein said immunostimulatory nucleic acid is an oligodeoxynucleotide (ODN) having at least one methylated CpG dinucleotide.

13. An improved method of inducing antibody dependent cellular cytotoxicity against a target cell in a mammalian subject, said method comprising:
a) activating the subject's NK cells ex vivo or in vivo with a cationic liposome comprising an immunostimulatory nucleic acid; and
b) opsonizing said target cell in vivo with a therapeutic antibody directed against a target cell antigen;
wherein said activated NK cells bind to the Fc portion of said therapeutic antibody in vivo.

14. The method according to claim 13, wherein said immunostimulatory nucleic acid is an oligodeoxynucleotide (ODN) having at least one methylated CpG dinucleotide.

15. The method according to claim 13, wherein said target cell is a tumor cell and said target cell antigen is a tumor-associated antigen.

16. A method of lysing tumor cells, comprising administering to a patient having said tumor cells a therapeutic antibody and a cationic liposome comprising an immunostimulatory nucleic acid, wherein said therapeutic antibody is directed to an antigen associated with said tumor cell and said cationic liposome mobilizes and activates patient NK cells in vivo for effectuating antibody dependent cellular cytotoxicity.

17. The method according to claim 16, wherein said immunostimulatory nucleic acid is an oligodeoxynucleotide (ODN) having at least one methylated CpG dinucleotide.

18. The method according to claim 16, wherein said cationic liposome is administered prior to said therapeutic antibody.

19. An improved method of treating a cancer patient with monoclonal antibodies directed to tumor-associated antigens, the improvement comprising pre-treating said patient with a cationic liposome comprising an immunostimulatory nucleic acid, wherein said pretreatment results in the mobilization and activation of patient NK cells for effectuating antibody dependent cellular cytotoxicity.

20. The method according to claim 19, wherein said immunostimulatory nucleic acid is an oligodeoxynucleotide (ODN) having at least one methylated CpG dinucleotide.

21. The method according to claim 19, wherein said cancer is lymphoma and said monoclonal antibody is rituximab.

22. The method according to claim 19, wherein said cancer is breast cancer and said therapeutic antibody is trastuzumab.

23. Means for lysing a target cell in a mammalian subject, comprising:
a) means for activating the subject's NK cells; and
b) means for opsonizing said target cell;
wherein said activated NK cells bind to the opsonized cell in vivo.

24. Means according to claim 23, wherein said means for activating comprises an oligodeoxynucleotide (ODN) having at least one methylated CpG dinucleotide.

25. Means according to claim 23, wherein said means for opsonizing comprises an antibody directed to a surface membrane antigen on said target cell

26. Means according to claim 23, wherein said target cell is a tumor cell and said means for opsonizing binds to a tumor-associated antigen.

27. A kit suitable for lysing a target cell in a mammalian patient, comprising a therapeutic antibody directed to a target cell antigen and a cationic liposome comprising an immunostimulatory nucleic acid.

28. The kit according to claim 27, wherein said therapeutic antibody and said cationic liposome are provided in separate vials.

29. The kit according to claim 27, wherein said immunostimulatory nucleic acid is an oligodeoxynucleotide (ODN) having at least one methylated CpG dinucleotide.

30. The kit according to claim 27, wherein said target cell is a tumor cell and said target cell antigen is a tumor-associated antigen.


SEQUENCE LISTING (33 sequences)

SEQ ID NO: 1 - DNA, 20 bp, Artificial
  Synthetic
tccatgacgttcctgacgtt

SEQ ID NO: 2 - DNA, 16 bp, Homo sapiens
taacgttgaggggcat

SEQ ID NO: 3 - DNA, 16 bp, Artificial
  Synthetic
taagcatacggggtgt

SEQ ID NO: 4 - DNA, 16 bp, Artificial
  Synthetic
taangttgaggggcat

SEQ ID NO: 5 - DNA, 6 bp, Artificial
  Synthetic
aacgtt

SEQ ID NO: 6 - DNA, 24 bp, Artificial
  Synthetic
gatgctgtgtcggggtctccgggc

SEQ ID NO: 7 - DNA, 24 bp, Artificial
  Synthetic
tcgtcgttttgtcgttttgtcgtt

SEQ ID NO: 8 - DNA, 24 bp, Artificial
  Synthetic
tngtngttttgtngttttgtngtt

SEQ ID NO: 9 - DNA, 20 bp, Artificial
  Synthetic
tccaggacttctctcaggtt

SEQ ID NO: 10 - DNA, 18 bp, Artificial
  Synthetic
tctcccagcgtgcgccat

SEQ ID NO: 11 - DNA, 20 bp, Mus musculus
tgcatcccccaggccaccat

SEQ ID NO: 12 - DNA, 20 bp, Homo sapiens
gcccaagctggcatccgtca

SEQ ID NO: 13 - DNA, 20 bp, Homo sapiens
gcccaagctggcatccgtca

SEQ ID NO: 14 - DNA, 15 bp, Homo sapiens
ggtgctcactgcggc

SEQ ID NO: 15 - DNA, 16 bp, Homo sapiens
aaccgttgaggggcat

SEQ ID NO: 16 - DNA, 24 bp, Homo sapiens
tatgctgtgccggggtcttcgggc

SEQ ID NO: 17 - DNA, 18 bp, Artificial
  Synthetic
gtgccggggtcttcgggc

SEQ ID NO: 18 - DNA, 18 bp, Homo sapiens
ggaccctcctccggagcc

SEQ ID NO: 19 - DNA, 18 bp, Homo sapiens
tcctccggagccagactt

SEQ ID NO: 20 - DNA, 15 bp, Homo sapiens
aacgttgaggggcat

SEQ ID NO: 21 - DNA, 15 bp, Homo sapiens
ccgtggtcatgctcc

SEQ ID NO: 22 - DNA, 21 bp, Homo sapiens
cagcctggctcaccgccttgg

SEQ ID NO: 23 - DNA, 20 bp, mus musculus
cagccatggttccccccaac

SEQ ID NO: 24 - DNA, 20 bp, Artificial
  Synthetic
gttctcgctggtgagtttca

SEQ ID NO: 25 - DNA, 18 bp, Homo sapiens
tctcccagcgtgcgccat

SEQ ID NO: 26 - DNA, 15 bp, Homo sapiens
gtgctccattgatgc

SEQ ID NO: 27 - DNA, 33 bp, Homo sapiens
gaguucugaugaggccgaaaggccgaaagucug

SEQ ID NO: 28 - DNA, 6 bp, Artificial
  Synthetic
rrcgyy

SEQ ID NO: 29 - DNA, 15 bp, Artificial
  Synthetic
aacgttgaggggcat

SEQ ID NO: 30 - DNA, 16 bp, Artificial
  Synthetic
caacgttatggggaga

SEQ ID NO: 31 - DNA, 16 bp, Homo sapiens
taacgttgaggggcat

SEQ ID NO: 32 - DNA, 20 bp, Artificial
  Synthetic
tccatgangttcctgangtt

SEQ ID NO: 33 - DNA, 21 bp, Artificial
  Synthetic
ttccatgacgttcctgacgtt